(19) United States
(12) Patent Application Publication
(10) Pub. No.: US 2003/0162163 A1
(43) Pub. Date: Aug. 28, 2003

(54) Method of sterilizing heart valves

(75) Inventors: Wilson Burgess, Clifton, VA (US); William N. Drohan, Springfield, VA (US); Martin J. MacPhee, Montgomery Village, MD (US); David M. Mann, Gaithersburg, MD (US)

Correspondence Address: FLESHNER & KIM, LLP, P.O. Box 221200, Chantilly, VA, 20153-1200, US

(73) Assignee: Clearant, Inc.

(21) Appl. No.: 10/379,789

(22) Filed: Mar. 6, 2003

Related U.S. Application Data
(62) Division of application No. 10/024,043, filed on Dec. 21, 2001.

(51) Int. Cl. A01N 1/02; A01N 1/00

(52) U.S. Cl. 435/1.1

(57) ABSTRACT

Methods are disclosed for sterilizing heart valves to reduce the level of one or more active biological contaminants or pathogens therein, such as viruses, bacteria, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites. The methods involve sterilizing one or more heart valves with irradiation.

### BACKGROUND OF THE INVENTION

1. Field of the Invention

The present invention relates to methods for sterilizing heart valves to reduce the level of one or more active biological contaminants or pathogens therein, such as viruses, bacteria (including inter- and intracellular bacteria, such as mycoplasmas, ureaplasmas, nanobacteria, chlamydia, rickettsias), yeasts, molds, fungi, prions or similar agents responsible, alone or in combination, for transmissible spongiform encephalopathies (TSEs) and/or single or multicellular parasites. The present invention particularly relates to methods of sterilizing heart valves with irradiation, wherein the heart valves may subsequently be used in transplantation to replace diseased and/or otherwise defective heart valves in an animal.

2. Background of the Related Art

Many biological materials that are prepared for human, veterinary, diagnostic and/or experimental use may contain unwanted and potentially dangerous biological contaminants or pathogens, such as viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single-cell or multicellular parasites. Consequently, it is of utmost importance that any biological contaminant or pathogen in the biological material be inactivated before the product is used. This is especially critical when the material is to be administered directly to a patient, for example in blood transfusions, blood factor replacement therapy, organ transplants, and other forms of human and/or other animal therapy corrected or treated by intravenous, intramuscular or other forms of injection or introduction. This is also critical for the various biological materials that are prepared in media or via the culture of cells, or recombinant cells which contain various types of plasma and/or plasma derivatives or other biologic materials and which may be subject to mycoplasmal, prion, ureaplasmal, bacterial, viral and/or other biological contaminants or pathogens.

Most procedures for producing human compatible biological materials have involved methods that screen or test the biological materials for one or more particular biological contaminants or pathogens rather than removal or inactivation of the contaminant(s) or pathogen(s) from the biological material. The typical protocol for disposition of materials that test positive for a biological contaminant or pathogen simply is non-use/discarding of that material. Examples of screening procedures for contaminants include testing for a particular virus in human blood and tissues from donors. Such procedures, however, are not always reliable, and are not able to detect the presence of certain viruses, particularly those in very low numbers. This reduces the value, certainty, and safety of such tests in view of the consequences associated with a false negative result, which can be life threatening in certain cases, for example in the case of Acquired Immune Deficiency Syndrome (AIDS). Furthermore, in some instances it can take weeks, if not months, to determine whether or not the material is contaminated. Moreover, to date, there is no commercially available, reliable test or assay for identifying prions, ureaplasmas, mycoplasmas, and chlamydia within a biological material that is suitable for screening out potential donors or infected material (Advances in Contraception 10(4):309-315(1994)). This serves to heighten the need for an effective means of destroying prions, ureaplasmas, mycoplasmas, chlamydia, etc., within a biological material, while still retaining the desired activity of that material. Therefore, it would be desirable to apply techniques that would kill or inactivate contaminants or pathogens during and/or after manufacturing and/or harvesting the biological material.

The importance of ready availability of effective techniques is apparent regardless of the source of the biological material. All living cells and multi-cellular organisms can be infected with viruses and other pathogens. Thus, the products of unicellular natural or recombinant organisms or tissues virtually always carry a risk of pathogen contamination. In addition to the risk that the producing cells or cell cultures may be infected, the processing of these and other biological materials also creates opportunities for environmental contamination. The risks of infection are more apparent for multicellular natural and recombinant organisms, such as transgenic animals. Interestingly, even products from species as different from humans as transgenic plants carry risks, both due to processing contamination as described above, and from environmental contamination in the growing facilities, which may be contaminated by pathogens from the environment or infected organisms that co-inhabit the facility alone with the desired plants. For example, a crop of transgenic corn grown out doors, could be expected to be exposed to rodents such as mice during the growing season. Mice can harbor serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overflying or perching birds has the potential to transmit such serious pathogens as the causative agent for psittacosis. Thus, any biological material, regardless of its source, may harbor serious pathogens that must be removed or inactivated prior to administration of the material to a recipient human or other animal.

In conducting experiments to determine the ability of technologies to inactivate viruses, the actual viruses of concern are seldom utilized. This is a result of safety concerns for the workers conducting the tests, and the difficulty and expense associated with facilities for containment and waste disposal. In their place, model viruses of the same family and class are usually used. In general, it is acknowledged that the most difficult viruses to inactivate are those with an outer shell made up of proteins, and that among these, the most difficult to inactivate are those of the smallest size. This has been shown to be true for gamma irradiation and most other forms of radiation because these viruses' diminutive size is associated with a small genome. The magnitude of direct effects of radiation upon a molecule is directly proportional to the size of the molecule; that is, the larger the target molecule, the greater is the effect. As a corollary, it has been shown for gamma-irradiation that the smaller the viral genome, the higher is the radiation dose required to inactive it.

Among the viruses of concern for both human and animal-derived biological materials, the smallest, and thus most difficult to inactivate, belong to the family of Parvoviruses and the slightly larger protein-coated Hepatitis virus. In humans, the Parvovirus B19, and Hepatitis A are the agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and, by extension, that it will also kill the larger and less hardy viruses, such as HIV, CMV, Hepatitis B, Hepatitis C, and others.

More recent efforts have focussed on methods to remove or inactivate contaminants in products intended for use in humans and other animals. Such methods include heat treating, filtration and the addition of chemical inactivants or sensitizers to the product.

According to current standards of the U.S. Food and Drug Administration, heat treatment of biological materials may require heating to approximately 60° C. for a minimum of 10 hours, which can be damaging to sensitive biological materials. Indeed, heat inactivation can destroy 50% or more of the biological activity of certain biological materials.

Filtration involves filtering the product in order to physically remove contaminants. Unfortunately, this method may also remove products that have a high molecular weight. Further, in certain cases, small viruses may not be removed by the filter.

The procedure of chemical sensitization involves the addition of noxious agents which bind to the DNA/RNA of the virus, and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate. This procedure requires that unbound sensitizer be washed from products since the sensitizers are toxic, if not mutagenic or carcinogenic, and cannot be administered to a patient.

Irradiating a product with gamma radiation is another method of sterilizing a product. Gamma radiation is effective in destroying viruses and bacteria when given in high total doses (Keathly, et al., “Is There Life After Irradiation? Part 2,” BioPharm July-August, 1993, and Leitman, “Use of Blood Cell Irradiation in the Prevention of Post Transfusion Graft-vs-Host Disease,” Transfusion Science 10:219-239(1989)). The published literature in this area, however, teaches that gamma radiation can be damaging to radiation sensitive products, such as blood, blood products, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective.” Unfortunately, many sensitive biological materials, such as monoclonal antibodies (Mab), may lose viability and activity if subjected to freezing for irradiation purposes and then thawing prior to administration to a patient.

When the product to be sterilized is biological tissue that is to be transplanted, even greater sensitivity to irradiation or other sterilization method is often encountered. This greater sensitivity is the result of the molecular integration of the biochemical, physiological, and anatomical systems that is required for normal function of that biological tissue. Thus, special procedures are typically required to maintain the tight molecular integration that underpins normal function during and after transplantation of a biological tissue. Furthermore, such special procedures are required in addition to other considerations, such as histocompatibility (matching of HLA types, etc.) between donor and recipient, and including compatibility between species when there is inter-species (i.e., heterografting) transplantation.

Tissues and organs that may be used in transplantation are numerous. Non-limiting examples include heart, lung, liver, spleen, pancreas, heart valves, kidney, corneas, bone, joints, bone marrow, blood cells (red blood cells, leucocytes, lymphocytes, platelets, etc.), plasma, skin, fat, tendons, ligaments, hair, muscles, blood vessels (arteries, veins), teeth, gum tissue, fetuses, eggs (fertilized and not fertilized), eye lenses, and even hands. Active research may soon expand this list to permit transplantation of nerve cells, nerves, and other physiologically and anatomically complex and other tissues, including intestine, cartilage, entire limbs, and portions of brain.

As surgical techniques become more sophisticated, and as storage and preparation techniques improve, the demand for various kinds of transplantation may reasonably be expected to increase over current levels.

Another factor that may feed future transplantation demand is certain poor lifestyle choices in the population, including such factors as poor nutrition (including such trends as the increasing reliance on so-called fast foods and fried foods; insufficient intake of fruits, vegetables and true whole grains; and increased intake of high glycemic, low nutritional value foods, including pastas, breads, white rice, crackers, potato chips and other snack foods, etc.), predilections toward a sedentary lifestyle, and over-exposure to ultraviolet light in tanning booths and to sunlight. The increasing occurrence of such factors as these have resulted, for example, in increased incidences of obesity (which also exacerbates such conditions as arthritis and conditions with cartilage damage, as well as impairs wound healing, immune function, cancer risk, etc.), type II diabetes and polycystic ovary syndrome (high post prandial glucose values causing damage to such tissues as nerve, muscle, kidney, heart, liver, etc., causing tissue and organ damage even in persons who are not diabetic), many cancers, and hypertension and other cardiovascular conditions, such as strokes and Alzheimer's disease (recent data suggesting that Alzheimer's may be the result of a series of mini-strokes). Thus, poor lifestyle choices ultimately will increase demand for bone, cartilage, skin, blood vessels, nerves, and the specific tissues and organs so destroyed or damaged.

Infections comprise yet another factor in transplantation demand. Not only can bacterial and viral infections broadly damage the infected host tissue or organ, but they can also spread vascularly or by lymphatics to cause lymph vessel or vascular inflammation, and/or plaque build up that ultimately results in infarct (for example, stroke, heart attack, damaged or dead tissue in lung or other organ, etc.). In addition, there is an epidemic of infection by intracellular microbes for which reliable commercial tests are not available (for example, mycoplasma, ureaplasma, and chlamydia), for example, as a result of sexual contact, coughing, etc. [for example, more than 20% of sore throats in children are due to chlamydia (E. Normann, et al., “Chlamydia Pneumoniae in Children Undergoing Adenoidectomy,” Acta Paediatrica 90(2):126-129(2001))].

Some intravascular infectious agents, via the antibodies that are produced to fight them, result in attack of tissue having surface molecules that have a molecular structure similar to the structure of surface or other groups of the infectious agent. Such is the case with some Streptococci infections (antibodies produced against M proteins of Streptococci that cross-react with cardiac, joint and other tissues), for example, in which heart valve and other cardiac tissue may be attacked to cause reduced cardiac function, and which can result in death if the infection is not properly treated before extensive damage occurs. Another antibody mediated condition that can affect cardiac tissue, among other tissues/cells, is antiphospholipid antibody syndrome (APLA), in which antibodies are directed against certain phospholipids (cardiolipin) to produce a hypercoagulable state, thrombocytopenia, fetal loss, dementia, strokes, optic changes, Addison's disease, and skin rashes, among other symptoms. Heart valve vegetations and mitral regurgitation are common in intravascular infections, although heart valve destruction so extensive as to require valve replacement is rare.

Other intravascular infectious agents directly attack tissues and organs in/on which they establish colonies. Non-limiting examples include Staphylococci (including, for example, S. aureus, S. epidermidis, S. saprophyticus, among others), Chlamydia (including, for example, C. pneumoniae, among others), Streptococci (including, for example, the viridians group of Streptococci: S. sanguis, S. oralis (mitis), S. salivarius, S. mutans, and others; and other species of Streptococci, such as S. bovis and S. pyogenes), Enterococci (for example, E. faecalis and E. faecium, among others), various fungi, and the “HACEK” group of gram-negative bacilli (Haemophilus parainfluenzae, Haemophilus aphrophilus, Actinibacillus actnomycetemcomitans, Cardiobacterium hominis, Eikenella corrodens, and Kingella kingae), Neisseria gonorrhoeae, Clostridia sp., Listeria moncytogenes, Salmonella sp., Bacteroides fragilis, Escherichia coli, Proteus sp., mycoplasmas, ureaplasmas, various viruses (for example, cytomegalovirus, HIV, and herpes simplex virus), and Klebsiella-Enterobacter-Serratia sp., among others.

An exemplary study by Nystrom-Rosander, et al. may be cited for showing the presence of Chlamydia pneumoniae in sclerotic heart valves that required replacement as a result of the sclerosis. (C. Nystrom-Rosander, et al., “High Incidence of Chlamydia pneumoniae in Sclerotic Heart Valve of Patients Undergoing Aortic Valve Replacement” Scandinavian Journal of Infectious Disease 29:361-365 (1997).

Yet another factor in transplantation demand is drug use, particularly the use of illicit drugs, but also including inappropriate and sometimes illegal use of otherwise licit drugs (such as overuse of alcohol/alcoholism causing cirrhosis of the liver, and therefore requiring liver transplantation). Such drug use often strongly damages or even destroys sensitive tissues and organs such as kidney, liver, lung, heart, brain/nerves, and/or portions thereof. In addition, intravenous drug use greatly increases the odds of contracting intravascular infections by any one or more of the above-cited infectious agents (among many others), which infections can attack virtually any organ or portion thereof including any of the four heart valves: the tricuspid valve (located between the right atrium and the right ventricle), the mitral valve (located between the left atrium and the left ventricle), the pulmonary or pulmonic valve (located between the right ventricle and the pulmonary artery), and the aortic valve (located between the left ventricle and the aorta).

In view of the difficulties discussed above, there remains a need for methods of sterilizing biological materials that are effective for reducing the level of active biological contaminants or pathogens without an adverse effect on the material(s).

The above references are incorporated by reference herein where appropriate for appropriate teachings of additional or alternative details, features and/or technical background.

### SUMMARY OF THE INVENTION

An object of the invention is to solve at least the related art problems and disadvantages, and to provide at least the advantages described hereinafter.

Accordingly, it is an object of the present invention to provide methods of sterilizing heart valves by reducing the level of active biological contaminants or pathogens without adversely affecting the heart valve or other material. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for sterilizing one or more heart valves that are sensitive to radiation, the method comprising irradiating the one or more heart valves with radiation for a time effective to sterilize the one or more heart valves at a rate effective to sterilize the one or more heart valves and to protect the one or more heart valves from the radiation.

Another embodiment of the present invention is directed to a method for sterilizing one or more heart valves that are sensitive to radiation, comprising: (i) applying to the one or more heart valves at least one stabilizing process selected from the group consisting of: (a) adding to the one or more heart valves at least one stabilizer in an amount effective to protect the one or more heart valves from the radiation; (b) reducing the residual solvent content of the one or more heart valves to a level effective to protect the one or more heart valves from the radiation; (c) reducing the temperature of the one or more heart valves to a level effective to protect the one or more heart valves from the radiation; (d) reducing the oxygen content of the one or more heart valves to a level effective to protect the one or more heart valves from the radiation; (e) adjusting the pH of the one or more heart valves to a level effective to protect the one or more heart valves from the radiation; and (f) adding to the one or more heart valves at least one non-aqueous solvent in an amount effective to protect the one or more heart valves from the radiation; and (ii) irradiating the one or more heart valves with a suitable radiation at an effective rate for a time effective to sterilize the one or more heart valves.

Another embodiment of the present invention is directed to a method for sterilizing one or more heart valves that are sensitive to radiation, comprising: (i) applying to the one or more heart valves at least one stabilizing process selected from the group consisting of: (a) adding to the one or more heart valves at least one stabilizer; (b) reducing the residual solvent content of the one or more heart valves; (c) reducing the temperature of the one or more heart valves; (d) reducing the oxygen content of the one or more heart valves; (e) adjusting the pH of the one or more heart valves; and (f) adding to the one or more heart valves at least one non-aqueous solvent; and (ii) irradiating the one or more heart valves with a suitable radiation at an effective rate for a time effective to sterilize the one or more heart valves, wherein the at least one stabilizing process and the rate of irradiation are together effective to protect the one or more heart valves from the radiation.

Another embodiment of the present invention is directed to a method for sterilizing one or more heart valves that are sensitive to radiation, comprising: (i) applying to the one or more heart valves at least two stabilizing processes selected from the group consisting of: (a) adding to the one or more heart valves at least one stabilizer; (b) reducing the residual solvent content of the one or more heart valves; (c) reducing the temperature of the one or more heart valves; (d) reducing the oxygen content of the one or more heart valves; (e) adjusting the pH of the one or more heart valves; and (f) adding to the one or more heart valves at least one non-aqueous solvent; and (ii) irradiating the one or more heart valves with a suitable radiation at an effective rate for a time effective to sterilize the one or more heart valves, wherein the at least two stabilizing processes are together effective to protect the one or more heart valves from the radiation and further wherein the at least two stabilizing processes may be performed in any order.

The invention also comprises a composition comprising one or more heart valves and at least one stabilizer in an amount effective to preserve the one or more heart valves for their intended use following sterilization with radiation.

The invention also provides a composition comprising one or more heart valves wherein the residual solvent content of the one or more heart valves is at a level effective to preserve the one or more heart valves for their intended use following sterilization with radiation.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings wherein:

FIGS. 1(a)-1(d) show the effects of porcine heart valves gamma irradiated in the presence of polypropylene glycol 400 (PPG400) and, optionally, a scavenger.

FIGS. 2(a)-2(e) show the effects of gamma irradiation on porcine heart valve cusps in the presence of 50% DMSO and, optionally, a stabilizer, and in the presence of polypropylene glycol 400 (PPG400).

FIGS. 3(a)-3(e) show the effects of gamma irradiation on frozen porcine AV heart valves soaked in various solvents and irradiated to a total dose of 30 kGy at 1.584 kGy/hr at −20° C.

FIGS. 4(a)-4(h) show the effects of gamma irradiation on frozen porcine AV heart valves soaked in various solvents and irradiated to a total dose of 45 kGy at approximately 6 kGy/hr at −70° C.

### DETAILED DESCRIPTION OF PREFERRED EMBODIMENTS

A. Definitions

Unless defined otherwise, all technical and scientific terms used herein are intended to have the same meaning as is commonly understood by one of ordinary skill in the relevant art.

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “sterilize” is intended to mean a reduction in the level of at least one active biological contaminant or pathogen found in the biological material being treated according to the present invention.

As used herein, the term “non-aqueous solvent” is intended to mean any liquid other than water in which a biological material, such as one or more heart valves, may be dissolved or suspended or which may be disposed within a biological material, such as one or more heart valves, and includes both inorganic solvents and, more preferably, organic solvents. Illustrative examples of suitable non-aqueous solvents include, but are not limited to, the following: alkanes and cycloalkanes, such as pentane, 2-methylbutane (isopentane), heptane, hexane, cyclopentane and cyclohexane; alcohols, such as methanol, ethanol, 2-methoxyethanol, isopropanol, n-butanol, t-butyl alcohol, and octanol; esters, such as ethyl acetate, 2-methoxyethyl acetate, butyl acetate and benzyl benzoate; aromatics, such as benzene, toluene, pyridine, xylene; ethers, such as diethyl ether, 2-ethoxyethyl ether, ethylene glycol dimethyl ether and methyl t-butyl ether; aldehydes, such as formaldehyde and glutaraldehyde; ketones, such as acetone and 3-pentanone (diethyl ketone); glycols, including both monomeric glycols, such as ethylene glycol and propylene glycol, and polymeric glycols, such as polyethylene glycol (PEG) and polypropylene glycol (PPG), e.g., PPG 400, PPG 1200 and PPG 2000; acids and acid anhydrides, such as formic acid, acetic acid, trifluoroacetic acid, phosphoric acid and acetic anhydride; oils, such as cottonseed oil, peanut oil, culture media, polyethylene glycol, poppyseed oil, safflower oil, sesame oil, soybean oil and vegetable oil; amines and amides, such as piperidine, N,N-dimethylacetamide and N,N-deimethylformamide; dimethylsulfoxide (DMSO); nitriles, such as benzonitrile and acetonitrile; hydrazine; detergents, such as polyoxyethylenesorbitan monolaurate (Tween 20) and monooleate (Tween 80), Triton and sodium dodecyl sulfate; carbon disulfide; halogenated solvents, such as dichloromethane, chloroform, carbon tetrachloride, 1,2-dichlorobenzene, 1,2-dichloroethane, tetrachloroethylene and 1-chlorobutane; furans, such as tetrahydrofuran; oxanes, such as 1,4-dioxane; and glycerin/glycerol. Particularly preferred examples of suitable non-aqueous solvents include non-aqueous solvents which also function as stabilizers, such as ethanol and acetone.

As used herein, the term “biological contaminant or pathogen” is intended to mean a biological contaminant or pathogen that, upon direct or indirect contact with a biological material, may have a deleterious effect on the biological material or upon a recipient thereof. Such other biological contaminants or pathogens include the various viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites known to those of skill in the art to generally be found in or infect biological materials. Examples of other biological contaminants or pathogens include, but are not limited to, the following: viruses, such as human immunodeficiency viruses and other retroviruses, herpes viruses, filoviruses, circoviruses, paramyxovirises, cytomegalovirmses, hepatitis viruses (including hepatitis A, B, C, and D variants thereof, among others), pox viruses, toga viruses, Ebstein-Barr viruses and parvoviruses; bacteria, such as Escherichia, Bacillus, Campylobacter, Streptococcus and Staphylococcus; nanobacteria; parasites, such as Trypanosoma and malarial parasites, including Plasmodium species; yeasts; molds; fungi; mycoplasmas and ureaplasmas; chlamydia; rickettsias, such as Coxiella burnetti; and prions and similar agents responsible, alone or in combination, for one or more of the disease states known as transmissible spongiform encephalopathies (TSEs) in mammals, such as scrapie, transmissible mink encephalopathy, chronic wasting disease (generally observed in mule deer and elk), feline spongiform encephalopathy, bovine spongiform encephalopathy (mad cow disease), Creutzfeld-Jakob disease (including variant CJD), Fatal Familial Insomnia, Gerstmann-Straeussler-Scheinker syndrome, kuru and Alpers syndrome. As used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in the biological material and/or a recipient thereof.

As used herein, the term “a biologically compatible solution” is intended to mean a solution to which a biological material may be exposed, such as by being suspended or dissolved therein, and remain viable, i.e., retain its essential biological and physiological characteristics.

As used herein, the term “a biologically compatible buffered solution” is intended to mean a biologically compatible solution having a pH and osmotic properties (e.g., tonicity, osmolality and/or oncotic pressure) suitable for maintaining the integrity of the material(s) therein. Suitable biologically compatible buffered solutions typically have a pH between 2 and 8.5 and are isotonic or only moderately hypotonic or hypertonic. Biologically compatible buffered solutions are known and readily available to those of skill in the art.

As used herein, the term “stabilizer” is intended to mean a compound or material that, alone and/or in combination, reduces damage to the biological material being irradiated to a level that is insufficient to preclude the safe and effective use of the material. Illustrative examples of stabilizers that are suitable for use include, but are not limited to, the following, including structural analogs and derivatives thereof: antioxidants; free radical scavengers, including spin traps, such as tert-butyl-nitrosobutane (tNB), a-phenyl-tert-butylnitrone (PBN), 5,5-dimethylpyrroline-N-oxide (DMPO), tert-butylnitrosobenzene (BNB), a-(4-pyridyl-1-oxide)-N-tert-butylnitrone (4-POBN) and 3,5-dibromo-4-nitroso-benzenesulphonic acid (DBNBS); combination stabilizers, i.e., stabilizers which are effective at quenching both Type I and Type II photodynamic reactions; and ligands, ligand analogs, substrates, substrate analogs, modulators, modulator analogs, stereoisomers, inhibitors, and inhibitor analogs, such as heparin, that stabilize the molecule(s) to which they bind. Preferred examples of additional stabilizers include, but are not limited to, the following: fatty acids, including 6,8-dimercapto-octanoic acid (lipoic acid) and its derivatives and analogues (alpha, beta, dihydro, bisno and tetranor lipoic acid), thioctic acid, 6,8-dimercapto-octanoic acid, dihydrolopoate (DL-6,8-dithioloctanoic acid methyl ester), lipoamide, bisonor methyl ester and tetranor-dihydrolipoic acid, omega-3 fatty acids, omega-6 fatty acids, omega-9 fatty acids, furan fatty acids, oleic, linoleic, linolenic, arachidonic, eicosapentaenoic (EPA), docosahexaenoic (DHA), and palmitic acids and their salts and derivatives; carotenes, including alpha-, beta-, and gamma-carotenes; Co-Q10; xanthophylls; sucrose, polyhydric alcohols, such as glycerol, mannitol, inositol, and sorbitol; sugars, including derivatives and stereoisomers thereof, such as xylose, glucose, ribose, mannose, fructose, erythrose, threose, idose, arabinose, lyxose, galactose, allose, altrose, gulose, talose, and trehalose; amino acids and derivatives thereof, including both D- and L-forms and mixtures thereof, such as arginine, lysine, alanine, valine, leucine, isoleucine, proline, phenylalanine, glycine, serine, threonine, tyrosine, asparagine, glutamine, aspartic acid, histidine, N-acetylcysteine (NAC), glutamic acid, tryptophan, sodium capryl N-acetyl tryptophan, and methionine; azides, such as sodium azide; enzymes, such as Superoxide Dismutase (SOD), Catalase, and Δ4, Δ5 and Δ6 desaturases; uric acid and its derivatives, such as 1,3-dimethyluric acid and dimethylthiourea; allopurinol; thiols, such as glutathione and reduced glutathione and cysteine; trace elements, such as selenium, chromium, and boron; vitamins, including their precursors and derivatives, such as vitamin A, vitamin C (including its derivatives and salts such as sodium ascorbate and palmitoyl ascorbic acid) and vitamin E (and its derivatives and salts such as alpha-, beta-, gamma-, delta-, epsilon-, zeta-, and eta-tocopherols, tocopherol acetate and alpha-tocotrienol); chromanol-alpha-C6; 6-hydroxy-2,5,7,8-tetramethylchroma-2 carboxylic acid (Trolox) and derivatives; extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS); 1,2-dithiane-4,5-diol; reducing substances, such as butylated hydroxyanisole (BHA) and butylated hydroxytoluene (BHT); cholesterol, including derivatives and its various oxidized and reduced forms thereof, such as low density lipoprotein (LDL), high density lipoprotein (HDL), and very low density lipoprotein (VLDL); probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); proteins, such as albumin, and peptides of two or more amino acids, any of which may be either naturally occurring amino acids, i.e., L-amino acids, or non-naturally occurring amino acids, i.e., D-amino acids, and mixtures, derivatives, and analogs thereof, including, but not limited to, arginine, lysine, alanine, valine, leucine, isoleucine, proline, phenylalanine, glycine, histidine, glutamic acid, tryptophan (Trp), serine, threonine, tyrosine, asparagine, glutamine, aspartic acid, cysteine, methionine, and derivatives thereof, such as N-acetylcysteine (NAC) and sodium capryl N-acetyl tryptophan, as well as homologous dipeptide stabilizers (composed of two identical amino acids), including such naturally occurring amino acids, as Gly-Gly (glycylglycine) and Trp-Trp, and heterologous dipeptide stabilizers (composed of different amino acids), such as carnosine (β-alanyl-histidine), anserine (β-alanyl-methylhistidine), and Gly-Trp; and flavonoids/flavonols, such as diosmin, quercetin, rutin, silybin, silidianin, silicristin, silymarin, apigenin, apiin, chrysin, morin, isoflavone, flavoxate, gossypetin, myricetin, biacalein, kaempferol, curcumin, proanthocyanidin B2-3-O-gallate, epicatechin gallate, epigallocatechin gallate, epigallocatechin, gallic acid, epicatechin, dihydroquercetin, quercetin chalcone, 4,4′-dihydroxy-chalcone, isoliquiritigenin, phloretin, coumestrol, 4′,7-dihydroxy-flavanone, 4′,5-dihydroxy-flavone, 4′,6-dihydroxy-flavone, luteolin, galangin, equol, biochanin A, daidzein, formononetin, genistein, amentoflavone, bilobetin, taxifolin, delphinidin, malvidin, petunidin, pelargonidin, malonylapliin, pinosylvin, 3-methoxyapigenin, leucodelphinidin, dihydrokaempferol, apigenin 7-O-glucoside, pycnogenol, aminoflavone, purpurogallin fisetin, 2′,3′-dihydroxyflavone, 3-hydroxyflavone, 3′,4′-dihydroxyflavone, catechin, 7-flavonoxyacetic acid ethyl ester, catechin, hesperidin, and naringin. Particularly preferred examples include single stabilizers or combinations of stabilizers that are effective at quenching both Type I and Type II photodynamic reactions, and volatile stabilizers, which can be applied as a gas and/or easily removed by evaporation, low pressure, and similar methods.

As used herein, the term “residual solvent content” is intended to mean the amount or proportion of freely-available liquid in the biological material. Freely-available liquid means the liquid, such as water and/or an organic solvent (e.g., ethanol, isopropanol, polyethylene glycol, etc.), present in the biological material being sterilized that is not bound to or complexed with one or more of the non-liquid components of the biological material. Freely-available liquid includes intracellular water and/or other solvents. The residual solvent contents related as water referenced herein refer to levels determined by the FDA approved, modified Karl Fischer method (Meyer and Boyd, Analytical Chem., 31:215-219, 1959; May, et al., J. Biol. Standardization, 10:249-259, 1982; Centers for Biologics Evaluation and Research, FDA, Docket No. 89D-0140, 83-93; 1990) or by near infrared spectroscopy. Quantitation of the residual levels of water or other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “sensitizer” is intended to mean a substance that selectively targets viruses, bacteria, in both vegetative and spore states, (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); inactines and their derivatives and analogs; angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphyrins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide. In addition, atoms which bind to prions, and thereby increase their sensitivity to inactivation by radiation, may also be used. An illustrative example of such an atom would be the Copper ion, which binds to the prion protein and, with a Z number higher than the other atoms in the protein, increases the probability that the prion protein will absorb energy during irradiation, particularly gamma irradiation.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “E-beam” irradiation that involves their production via a machine. Visible light, both mono- and polychromatic, is produced by machines and may, in practice, be combined with invisible light, such as infrared and UV, that is produced by the same machine or a different machine.

As used herein, the term “to protect” is intended to mean to reduce any damage to the biological material being irradiated, that would otherwise result from the irradiation of that material, to a level that is insufficient to preclude the safe and effective use of the material following irradiation. In other words, a substance or process “protects” a biological material from radiation if the presence of that substance or carrying out that process results in less damage to the material from irradiation than in the absence of that substance or process. Thus, a biological material may be used safely and effectively after irradiation in the presence of a substance or following performance of a process that “protects” the material, but could not be used with as great a degree of safety or as effectively after irradiation under identical conditions but in the absence of that substance or the performance of that process.

As used herein, an “acceptable level” of damage may vary depending upon certain features of the particular method(s) of the present invention being employed, such as the nature and characteristics of the particular biological material and/or non-aqueous solvent(s) being used, and/or the intended use of the biological material being irradiated, and can be determined empirically by one skilled in the art. An “unacceptable level” of damage would therefore be a level of damage that would preclude the safe and effective use of the biological material being sterilized. The particular level of damage in a given biological material may be determined using any of the methods and techniques known to one skilled in the art.

B. Particularly Preferred Embodiments

A first preferred embodiment of the present invention is directed to a method for sterilizing one or more heart valves that are sensitive to radiation, the method comprising irradiating the one or more heart valves with radiation for a time effective to sterilize the one or more heart valves at a rate effective to sterilize the one or more heart valves and to protect the one or more heart valves from the radiation.

A second preferred embodiment of the present invention is directed to a method for sterilizing one or more heart valves that are sensitive to radiation, comprising: (i) applying to the one or more heart valves at least one stabilizing process selected from the group consisting of: (a) adding to the one or more heart valves at least one stabilizer in an amount effective to protect the one or more heart valves from the radiation; (b) reducing the residual solvent content of the one or more heart valves to a level effective to protect the one or more heart valves from the radiation; (c) reducing the temperature of the one or more heart valves to a level effective to protect the one or more heart valves from the radiation; (d) reducing the oxygen content of the one or more heart valves to a level effective to protect the one or more heart valves from the radiation; (e) adjusting the pH of the one or more heart valves to a level effective to protect the one or more heart valves from the radiation; and (f) adding to the one or more heart valves at least one non-aqueous solvent in an amount effective to protect the one or more heart valves from the radiation; and (ii) irradiating the one or more heart valves with a suitable radiation at an effective rate for a time effective to sterilize the one or more heart valves.

A third preferred embodiment of the present invention is directed to a method for sterilizing one or more heart valves that are sensitive to radiation, comprising: (i) applying to the one or more heart valves at least one stabilizing process selected from the group consisting of: (a) adding to the one or more heart valves at least one stabilizer; (b) reducing the residual solvent content of the one or more heart valves; (c) reducing the temperature of the one or more heart valves; (d) reducing the oxygen content of the one or more heart valves; (e) adjusting the pH of the one or more heart valves; and (f) adding to the one or more heart valves at least one non-aqueous solvent; and (ii) irradiating the one or more heart valves with a suitable radiation at an effective rate for a time effective to sterilize the one or more heart valves, wherein the at least one stabilizing process and the rate of irradiation are together effective to protect the one or more heart valves from the radiation.

A fourth preferred embodiment of the present invention is directed to a method for sterilizing one or more heart valves that are sensitive to radiation, comprising: (i) applying to the one or more heart valves at least two stabilizing processes selected from the group consisting of: (a) adding to the one or more heart valves at least one stabilizer; (b) reducing the residual solvent content of the one or more heart valves; (c) reducing the temperature of the one or more heart valves; (d) reducing the oxygen content of the one or more heart valves; (e) adjusting the pH of the one or more heart valves; and (f) adding to the one or more heart valves at least one non-aqueous solvent; and (ii) irradiating the one or more heart valves with a suitable radiation at an effective rate for a time effective to sterilize the one or more heart valves, wherein the at least two stabilizing processes are together effective to protect the one or more heart valves from the radiation and further wherein the at least two stabilizing processes may be performed in any order.

Another preferred embodiment of the present invention is directed to a composition comprising one or more heart valves and at least one stabilizer in an amount effective to preserve the one or more heart valves for their intended use following sterilization with radiation.

Another preferred embodiment of the present invention is directed to a composition comprising one or more heart valves, wherein the residual solvent content of the one or more heart valves is at a level effective to preserve the one or more heart valves for their intended use following sterilization with radiation.

The non-aqueous solvent is preferably a non-aqueous solvent that is not prone to the formation of free-radicals upon irradiation, and more preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

According to certain embodiments of the present invention, the one or more heart valves may contain a mixture of water and a non-aqueous solvent, such as ethanol and/or acetone. In such embodiments, the non-aqueous solvent(s) is (are) preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are also stabilizers, such as ethanol and acetone.

According to certain methods of the present invention, a stabilizer is added prior to irradiation of the one or more heart valves with radiation. This stabilizer is preferably added to the one or more heart valves in an amount that is effective to protect the one or more heart valves from the radiation. Alternatively, the stabilizer is added to the one or more heart valves in an amount that, together with a non-aqueous solvent, is effective to protect the one or more heart valves from the radiation. Suitable amounts of stabilizer may vary depending upon certain features of the particular method(s) of the present invention being employed, such as the particular stabilizer being used and/or the nature and characteristics of the particular one or more heart valves being irradiated and/or its intended use, and can be determined empirically by one skilled in the art.

According to certain methods of the present invention, the residual solvent content of the one or more heart valves is reduced prior to irradiation of the one or more heart valves with radiation. The residual solvent content is preferably reduced to a level that is effective to protect the one or more heart valves from the radiation. Suitable levels of residual solvent content may vary depending upon certain features of the particular method(s) of the present invention being employed, such as the nature and characteristics of the particular one or more heart valves being irradiated and/or its intended use, and can be determined empirically by one skilled in the art. There may be heart valves for which it is desirable to maintain the residual solvent content to within a particular range, rather than a specific value.

According to certain embodiments of the present invention, when the one or more heart valves also contain water, the residual solvent (water) content of one or more heart valves may be reduced by dissolving or suspending the one or more heart valves in a non-aqueous solvent that is capable of dissolving water. Preferably, such a non-aqueous solvent is not prone to the formation of free-radicals upon irradiation and has little or no dissolved oxygen or other gas(es) that is (are) prone to the formation of free-radicals upon irradiation.

While not wishing to be bound by any theory of operability, it is believed that the reduction in residual solvent content reduces the degrees of freedom of the one or more heart valves, reduces the number of targets for free radical generation and may restrict the diffusability of these free radicals. Similar results might therefore be achieved by lowering the temperature of the one or more heart valves below their eutectic point(s) or below their freezing point(s), or by vitrification to likewise reduce the degrees of freedom of the one or more heart valves. These results may permit the use of a higher rate and/or dose of radiation than might otherwise be acceptable. Thus, the methods described herein may be performed at any temperature that doesn't result in unacceptable damage to the one or more heart valves, i e., damage that would preclude the safe and effective use of the one or more heart valves. Preferably, the methods described herein are performed at ambient temperature or below ambient temperature, such as below the eutectic point(s) or freezing point(s) of the one or more heart valves being irradiated.

In certain embodiments of the present invention, the desired residual solvent content of a particular heart valve may be found to lie within a range, rather than at a specific point. Such a range for the preferred residual solvent content of a particular heart valve may be determined empirically by one skilled in the art.

The residual solvent content of the one or more heart valves may be reduced by any of the methods and techniques known to those skilled in the art for reducing solvent from one or more heart valves without producing an unacceptable level of damage to the one or more heart valves. Such methods include, but are not limited to, lyophilization, drying, concentration, addition of alternative solvents, evaporation, chemical extraction and vitrification.

A particularly preferred method for reducing the residual solvent content of one or more heart valves is lyophilization.

Another particularly preferred method for reducing the residual solvent content of one or more heart valves is vitrification, which may be accomplished by any of the methods and techniques known to those skilled in the art, including the addition of solute and or additional solutes, such as sucrose, to raise the eutectic point(s) of the one or more heart valves, followed by a gradual application of reduced pressure to the one or more heart valves in order to remove the residual solvent. The resulting glassy material will then have a reduced residual solvent content.

According to certain methods of the present invention, the one or more heart valves to be sterilized may be immobilized upon or attached to a solid surface by any means known and available to one skilled in the art. For example, the one or more heart valves to be sterilized may be attached to a biological or non-biological substrate.

The radiation employed in the methods of the present invention may be any radiation effective for the sterilization of the one or more heart valves being treated. The radiation may be corpuscular, including E-beam radiation. Preferably the radiation is electromagnetic radiation, including x-rays, infrared, visible light, UV light and mixtures of various wavelengths of electromagnetic radiation. A particularly preferred form of radiation is gamma radiation.

According to the methods of the present invention, the one or more heart valves are irradiated with the radiation at a rate effective for the sterilization of the one or more heart valves, while not producing an unacceptable level of damage to the one or more heart valves. Suitable rates of irradiation may vary depending upon certain features of the methods of the present invention being employed, such as the nature and characteristics of the particular heart valves, which may contain a non-aqueous solvent, being irradiated, the particular form of radiation involved, and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (≦3 kGy/hour) and high (>3 kGy/hour) rates may be utilized in the methods described herein to achieve such results. The rate of irradiation is preferably selected to optimize the recovery of the one or more heart valves while still sterilizing the one or more heart valves. Although reducing the rate of irradiation may serve to decrease damage to the one or more heart valves, it will also result in longer irradiation times being required to achieve a particular desired total dose. A higher dose rate may therefore be preferred in certain circumstances, such as to minimize logistical issues and costs, and may be possible particularly when used in accordance with the methods described herein for protecting heart valves from irradiation.

According to a particularly preferred embodiment of the present invention, the rate of irradiation is not more than about 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr.

According to another particularly preferred embodiment of the present invention, the rate of irradiation is at least about 3.0 kGy/hr, more preferably at least about 6 kGy/hr, even more preferably at least about 16 kGy/hr, even more preferably at least about 30 kGy/hr and most preferably at least about 45 kGy/hr or greater.

According to the methods of the present invention, the one or more heart valves to be sterilized are irradiated with the radiation for a time effective for the sterilization of the one or more heart valves. Combined with irradiation rate, the appropriate irradiation time results in the appropriate dose of irradiation being applied to the one or more heart valves. Suitable irradiation times may vary depending upon the particular form and rate of radiation involved and/or the nature and characteristics of the particular one or more heart valves being irradiated. Suitable irradiation times can be determined empirically by one skilled in the art.

According to the methods of the present invention, the one or more heart valves to be sterilized are irradiated with radiation up to a total dose effective for the sterilization of the one or more heart valves, while not producing an unacceptable level of damage to those one or more heart valves. Suitable total doses of radiation may vary depending upon certain features of the methods of the present invention being employed, such as the nature and characteristics of the particular one or more heart valves being irradiated, the particular form of radiation involved, and/or the particular biological contaminants or pathogens being inactivated. Suitable total doses of radiation can be determined empirically by one skilled in the art. Preferably, the total dose of radiation is at least 25 kGy, more preferably at least 45 kGy, even more preferably at least 75 kGy, and still more preferably at least 100 kGy or greater, such as 150 kGy or 200 kGy or greater.

The particular geometry of the one or more heart valves being irradiated, such as the thickness and distance from the source of radiation, may be determined empirically by one skilled in the art. A preferred embodiment is a geometry that provides for an even rate of irradiation throughout the preparation of one or more heart valves. A particularly preferred embodiment is a geometry that results in a short path length for the radiation through the preparation, thus minimizing the differences in radiation dose between the front and back of the preparation. This may be further minimized in some preferred geometries, particularly those wherein the preparation of one or more heart valves has a relatively constant radius about its axis that is perpendicular to the radiation source and by the utilization of a means of rotating the preparation of one or more heart valves about said axis.

Similarly, according to certain methods of the present invention, an effective package for containing the preparation of one or more heart valves during irradiation is one which combines stability under the influence of irradiation, and which minimizes the interactions between the package of one or more heart valves and the radiation. Preferred packages maintain a seal against the external environment before, during and post-irradiation, and are not reactive with the preparation of one or more heart valves within, nor do they produce chemicals that may interact with the preparation of one or more heart valves within. Particularly preferred examples include but are not limited to containers that comprise glasses stable when irradiated, stoppered with stoppers made of rubber or other suitable materials that is relatively stable during radiation and liberates a minimal amount of compounds from within, and sealed with metal crimp seals of aluminum or other suitable materials with relatively low Z numbers. Suitable materials can be determined by measuring their physical performance, and the amount and type of reactive leachable compounds post-irradiation, and by examining other characteristics known to be important to the containment of such biological materials as heart valves empirically by one skilled in the art.

According to certain methods of the present invention, an effective amount of at least one sensitizing compound may optionally be added to the one or more heart valves prior to irradiation, for example to enhance the effect of the irradiation on the biological contaminant(s) or pathogen(s) therein, while employing the methods described herein to minimize the deleterious effects of irradiation upon the one or more heart valves. Suitable sensitizers are known to those skilled in the art, and include psoralens and their derivatives and inactines and their derivatives.

According to the methods of the present invention, the irradiation of the one or more heart valves may occur at any temperature that is not deleterious to the one or more heart valves being sterilized. According to one preferred embodiment, the one or more heart valves are irradiated at ambient temperature. According to an alternate preferred embodiment, the one or more heart valves are irradiated at reduced temperature, i.e., a temperature below ambient temperature, such as 0° C., −20° C., −40° C., −60° C., −78° C. or −196° C. According to this embodiment of the present invention, the one or more heart valves are preferably irradiated at or below the freezing or eutectic point(s) of the one or more heart valves or the residual solvent therein. According to another alternate preferred embodiment, the one or more heart valves are irradiated at elevated temperature, i.e., a temperature above ambient temperature, such as 37° C., 60° C., 72° C. or 80° C. While not wishing to be bound by any theory, the use of elevated temperature may enhance the effect of irradiation on the biological contaminant(s) or pathogen(s) and therefore allow the use of a lower total dose of radiation.

Most preferably, the irradiation of the one or more heart valves occurs at a temperature that protects the preparation of one or more heart valves from radiation. Suitable temperatures can be determined empirically by one skilled in the art.

In certain embodiments of the present invention, the temperature at which irradiation is performed may be found to lie within a range, rather than at a specific point. Such a range for the preferred temperature for the irradiation of a particular heart valve may be determined empirically by one skilled in the art.

According to the methods of the present invention, the irradiation of the one or more heart valves may occur at any pressure which is not deleterious to the one or more heart valves being sterilized. According to one preferred embodiment, the one or more heart valves are irradiated at elevated pressure. More preferably, the one or more heart valves are irradiated at elevated pressure due to the application of sound waves or the use of a volatile. While not wishing to be bound by any theory, the use of elevated pressure may enhance the effect of irradiation on the biological contaminant(s) or pathogen(s) and/or enhance the protection afforded by one or more stabilizers, and therefore allow the use of a lower total dose of radiation. Suitable pressures can be determined empirically by one skilled in the art.

Generally, according to the methods of the present invention, the pH of the one or more heart valves undergoing sterilization is about 7. In some embodiments of the present invention, however, the one or more heart valves may have a pH of less than 7, preferably less than or equal to 6, more preferably less than or equal to 5, even more preferably less than or equal to 4, and most preferably less than or equal to 3. In alternative embodiments of the present invention, the one or more heart valves may have a pH of greater than 7, preferably greater than or equal to 8, more preferably greater than or equal to 9, even more preferably greater than or equal to 10, and most preferably greater than or equal to 11. According to certain embodiments of the present invention, the pH of the preparation of one or more heart valves undergoing sterilization is at or near the isoelectric point of one of the components of the one or more heart valves. Suitable pH levels can be determined empirically by one skilled in the art.

Similarly, according to the methods of the present invention, the irradiation of the one or more heart valves may occur under any atmosphere that is not deleterious to the one or more heart valves being treated. According to one preferred embodiment, the one or more heart valves are held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the one or more heart valves are held under vacuum while being irradiated. According to a particularly preferred embodiment of the present invention, the one or more heart valves (lyophilized, liquid or frozen) are stored under vacuum or an inert atmosphere (preferably a noble gas, such as helium or argon, more preferably a higher molecular weight noble gas, and most preferably argon) prior to irradiation. According to an alternative preferred embodiment of the present invention, the one or more heart valves are held under low pressure, to decrease the amount of gas, particularly oxygen and nitrogen, dissolved in the liquid, prior to irradiation, either with or without a prior step of solvent reduction, such as lyophilization. Such degassing may be performed using any of the methods known to one skilled in the art.

In another preferred embodiment, where the one or more heart valves contain oxygen or other gases dissolved within the one or more heart valves or within their container or associated with them, the amount of these gases within or associated with the preparation of one or more heart valves may be reduced by any of the methods and techniques known and available to those skilled in the art, such as the controlled reduction of pressure within a container (rigid or flexible) holding the preparation of one or more heart valves to be treated or by placing the preparation of one or more heart valves in a container of approximately equal volume.

In certain embodiments of the present invention, when the one or more heart valves to be treated contain an aqueous or non-aqueous solvent, at least one stabilizer is introduced according to any of the methods and techniques known and available to one skilled in the art, including soaking the heart valve tissue in a solution containing the stabilizer(s), preferably under pressure, at elevated temperature and/or in the presence of a penetration enhancer, such as dimethylsulfoxide. Other methods of introducing at least one stabilizer into heart valve tissue include, but are not limited to, applying a gas containing the stabilizer(s), preferably under pressure and/or at elevated temperature, injection of the stabilizer(s) or a solution containing the stabilizer(s) directly into the heart valve tissue, placing the heart valve tissue under reduced pressure and then introducing a gas or solution containing the stabilizer(s), dehydrating the heart valve tissue and rehydrating the heart valve tissue with a solution containing at least one stabilizer, and combinations of two or more of these methods. One or more sensitizers may also be introduced into heart valve tissue according to such methods.

It will be appreciated that the combination of one or more of the features described herein may be employed to further minimize undesirable effects upon the one or more heart valves caused by irradiation, while maintaining adequate effectiveness of the irradiation process on the biological contaminant(s) or pathogen(s). For example, in addition to the use of a stabilizer, a particular heart valve may also be lyophilized, held at a reduced temperature and kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D₃₇ value. The desirable components of a heart valve may also be considered to have a D₃₇ value equal to the dose of radiation required to eliminate all but 37% of their desirable biological and physiological characteristics.

In accordance with certain preferred methods of the present invention, the sterilization of one or more heart valves are conducted under conditions that result in a decrease in the D₃₇ value of the biological contaminant or pathogen without a concomitant decrease in the D₃₇ value of the one or more heart valves. In accordance with other preferred methods of the present invention, the sterilization of one or more heart valves is conducted under conditions that result in an increase in the D₃₇ value of the heart valve material. In accordance with the most preferred methods of the present invention, the sterilization of one or more heart valves is conducted under conditions that result in a decrease in the D₃₇ value of the biological contaminant or pathogen and a concomitant increase in the D₃₇ value of the one or more heart valves.

### EXAMPLES

The following examples are illustrative, but not limiting, of the present invention. Other suitable modifications and adaptations are of the variety normally encountered by those skilled in the art and are fully within the spirit and scope of the present invention. For example, heart valves from animal species other than pig, such as bovine or human, are encompassed by this technology, as are heart valves from transgenic mammals. In addition, heart valves prepared/modified by practice of the present invention may be used for transplantation into any animal, particularly into mammals. Furthermore, the principles of the technology of the present invention may be practiced on animal tissues and organs other than heart valves. Unless otherwise noted, all irradiation was accomplished using a ⁶⁰Co source.

### Example 1 [1071001.esm.0048]

In this experiment, porcine heart valves were gamma irradiated in the presence of polypropylene glycol 400 (PPG400) and, optionally, a scavenger, to a total dose of 30 kGy (1.584 kGy/hr at −20° C.).

Materials:

Tissue—Porcine Pulmonary Valve (PV) Heart valves were harvested prior to use and stored.

Tissue Preparation Reagents—

Polypropylene Glycol 400. Fluka: cat#81350, lot#386716/1

Trolox C. Aldrich: cat#23,881-3, lot#02507TS

Coumaric Acid. Sigma: cat#C-9008, lot#49H3600

n-Propyl Gallate. Sigma: cat#P-3130, lot#117H0526

α-Lipoic Acid. CalBiochem: cat#437692, lot#B34484

Dulbecco's PBS. Gibco BRL: cat#14190-144, lot#1095027

2.0 ml Screw Cap tubes. VWR Scientific Products: cat#20170-221, lot#0359

Tissue Hydrolysis Reagents—

Nerl H₂O. NERL Diagnostics: cat#9800-5, lot#03055151

Acetone. EM Science: cat#AX0125-5,lot#37059711

6 N constant boiling HCl. Pierce: cat#24309, lot#BA42184

Int-Pyd (Acetylated Pyridinoline) HPLC Internal Standard. Metra Biosystems Inc.: cat#8006, lot#9H142, expiration 2/2002, Store at ≦−20° C.

Hydrochloric Acid. VWR Scientific: cat#VW3110-3, lot#n/a

Heptafluorobutyric Acid (HFBA) Sigma: cat#H-7133, lot#20K3482 FW 214.0 store at 2-8° C.

SP-Sephadex C-25 resin. Pharmacia: cat#17-0230-01, lot#247249 (was charged with NaCl as per manufacturer suggestion)

Hydrolysis vials—10 mm×100 mm vacuum hydrolysis tubes. Pierce: cat#29560, lot #BB627281

Heating module—Pierce, Reacti-therm.: Model #18870, S/N 1125000320176

Savant—Savant Speed Vac System:

Speed Vac Model SC110, model #SC110-120, serial #SC110-SD171002-1H

a. Refrigerated Vapor Trap Model RVT100, model #RVT100-120V, serial #RVT100-58010538-1B

b. Vacuum pump, VP 100 Two Stage Pump Model VP100, serial #93024

Column—Phenomenex, Luna 5 μC18(2) 100 Å, 4.6×250 mm. Part #00G-4252-E0, S/N#68740-25, B/N#5291-29

HPLC System:

Shimadzu System Controller SCL-10A

Shimadzu Automatic Sample Injector SIL-10A (50 μl loop)

Shimadzu Spectrofluorometric Detector RF-10A

Shimadzu Pumps LC-10AD

Software—Class-VP version 4.1

Low-binding tubes—MiniSorp 100×15 Nunc-Immunotube. Batch #042950, cat#468608

Methods

A. Preparation of Stabilizer Solutions:

Trolox C:

MW=250; therefore, 250 mg/ml needed for a 1M solution and 125 mg/ml for a 0.5M solution actual weight measured was 250.9 mg

250.9÷125 mg/ml=2.0 ml needed to make a 0.5M solution

The 0.5 M solution was not soluble; therefore an additional 2 ml of PPG was added. After water bath sonication at 25° C. and above for at least 30 minutes, Trolox C is soluble at 125 mM.

Coumaric Acid:

MW=164; therefore, 164 mg/ml needed for a 1M solution actual weight measured was 164.8 mg

164.8 mg÷164 mg/ml=1.0 ml needed to make a 1M solution

Water bath sonicated at 25° C. and above for approximately 15 minutes—not 100% soluble. An additional 1 ml PPG was added and further water bath sonicated.

n-Propyl Gallate:

MW=212.2; therefore, 212 mg/ml needed for a 1M and 106 mg/ml for a 0.5 M solution actual weight measured was 211.9 mg

211.9 mg÷106 mg/ml=2.0 ml needed to make a 0.5M solution

The 0.5M solution was soluble after a 20-30 minute water bath sonication.

1 M α-Lipoic Acid:

MW=206; therefore, 206 mg/ml needed for a 1M solution actual weight measured was 412 mg

412 mg÷206 mg/ml=2.0 ml needed to make a 1M solution

Very soluble after 10 minute water bath sonication.

Final Stocks of Scavengers:

125 mM Trolox C—4 ml

0.5 M Coumaric acid—2 ml

0.5 M n-Propyl Gallate—2 ml

1 M Lipoic Acid—2 ml

B. Treatment of Valves Prior to Gamma-Irradiation.

1. PV heart valves were thawed on wet ice.

2. Cusps were dissected out from each valve and pooled into 50 ml conical tubes containing cold Dulbecco's PBS.

3. Cusps were washed in PBS at 4° C. for approximately 1.5 hrs; changing PBS during that time a total of 6 times.

4. 2 cusps were placed in each of six 2 ml screw cap tube.

5. 1.2 ml of PPG were added to two tubes (one of these tubes was designated 0 kGy and the other tube was designated 30 kGy):

1.2 ml of 125 mM Trolox C in PPG were added to another tnvo tubes

1.2 ml of SCb stabilizer mixture—comprising of 1.5 ml 125 mM Trolox C, 300 μl 1 M Lipoic Acid, 600 μl 0.5 M Coumaric Acid and 600 μl 0.5 M n-Propyl Gallate (Final concentrations: 62.5 mM, 100 mM, 100 mM and 100 mM respectively) were added to the final two tubes.

6. Tubes were incubated at 4° C., with rocking for about 60 hours.

7. Stabilizer solutions and cusps were transferred into 2 ml glass vials for gamma-irradiation.

8. All vials were frozen on dry ice.

9. Control samples were kept in-house at −20° C.

C. Gamma-Irradiation of Tissue.

Samples were irradiated at a rate of 1.584 kGy/hr at −20° C. to a total dose of 30 kGy.

D. Processing Tissue for Hydrolysis/Extraction.

1. Since PPG is viscous, PBS was added to allow for easier transfer of material.

2. Each pair of cusps (2 per condition) were placed into a 50 ml Falcon tube filled with cold PBS and incubated on ice—inverting tubes periodically.

3. After one hour PBS was decanted from the tubes containing cusps in PPG/0 kGy and PPG/30 kGy and replenished with fresh cold PBS. For the PPG samples containing Trolox C or SCb stabilizer mixture, fresh 50 ml Falcon tubes filled with cold PBS were set-up and the cusps transferred.

4. An additional 3 washes were done.

5. One cusp was transferred into a 2 ml Eppendorf tube filled with cold PBS for extraction. The other cusp was set-up for hydrolysis.

E. Hydrolysis of Tissue.

1. Each cusp was washed 6× with acetone in an Eppendorf tube (approximately 1.5 ml/wash).

2. Each cusp was subjected to SpeedVac (with no heat) for approximately 15 minutes or until dry.

3. Samples were weighed, transferred to hydrolysis vials and 6 N HCl added at a volume of 20 mg tissue/ml HCl:

| Sample ID | Dry Weight (mg) | μl 6 N HCl |
|---|---|---|
| 1. PPG/0 | 6.49 | 325 |
| 2. PPG/30 | 7.26 | 363 |
| 3. PPG T/0 | 5.80 | 290 |
| 4. PPG T/30 | 8.20 | 410 |
| 5. PPG SCb/0 | 6.41 | 321 |
| 6. PPG SCb/30 | 8.60 | 430 |

4. Samples were hydrolyzed at 110° C. for approximately 23 hours.

5. Hydrolysates were transferred into Eppendorf tubes and centrifuged @12,000 rpm for 5 min.

6. Supernatent was then transferred into a clean Eppendorf.

7. 50 μl of hydrolysate was diluted in 8 ml Nerl H₂O (diluting HCl to approximately 38 mM).

8. Spiked in 200 μl of 2× int-pyd. Mixed by inversion. (For 1600 μl 2× int-pyd:160 μl 20× int-pyd+1440 μl Nerl H₂O.)

9. Samples were loaded onto SP-Sephadex C25 column (approximately 1×1 cm packed bed volume) that had been equilibrated in water. (Column was pre-charged with NaCl)

10. Loaded flow through once again over column.

11. Washed with 20 ml 150 mM HCl.

12. Eluted crosslinks with 5 ml 2 N HCl into a low binding tube.

13. Dried entire sample in Savant.

F. Analysis of Hydrolysates.

Set-up the following:

| Sample | μl | μl H₂O | μl HFBA |
|---|---|---|---|
| 1. PPG/0 kGy | 18 | 180 | 2 |
| 2. PPG/30 kGy | 59 | 139 | 2 |
| 3. PPG T/0 kGy | 67 | 171 | 2 |
| 4. PPG T/30 kGy | 64 | 134 | 2 |
| 5. PPG SCb/0 kGy | 10 | 188 | 2 |
| 6. PPG SCb/30 kGy | 32 | 166 | 2 |

Results:

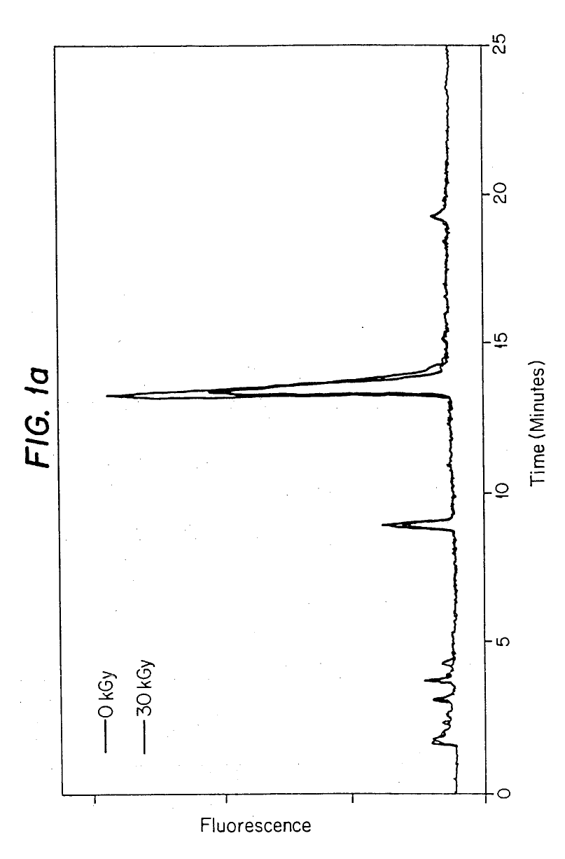

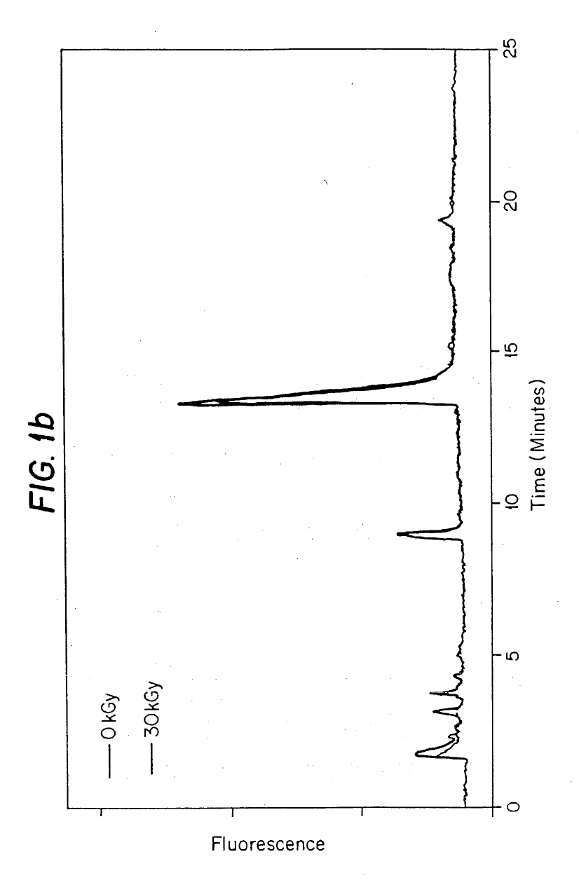

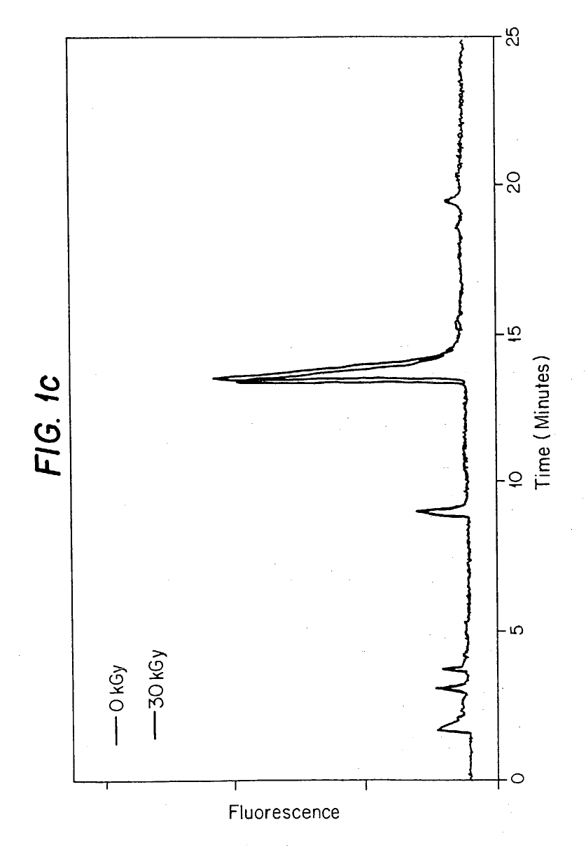

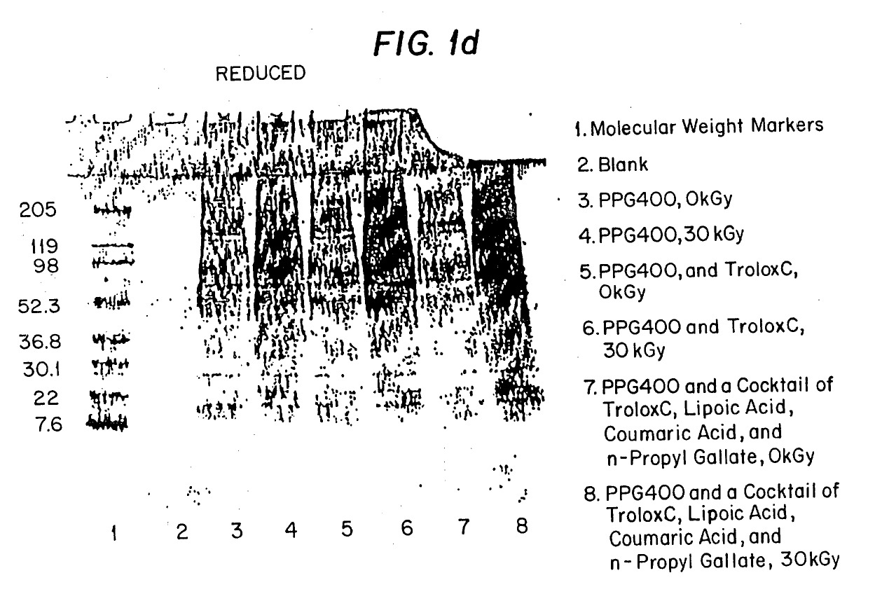

The HPLC results are shown in FIGS. 1A-1C. In the presence of PPG 400, the results were nearly identical whether the heart valve had been irradiated or not. The addition of a single stabilizer (trolox C) or a stabilizer mixture produced even more effective results. The gel analysis, shown in FIG. 1D, confirmed the effectiveness of the protection provided by these conditions.

### Example 2 [061501.esm.0042/062601alm068]

In this experiment, the effects of gamma irradiation were determined on porcine heart valve cusps in the presence of 50% DMSO and, optionally, a stabilizer, and in the presence of polypropylene glycol 400 (PPG400).

Preparation of Tissue for Irradiation:

1. 5 vials of PV and 3 vials of atrial valves (AV) were thawed on ice.

2. Thaw media was removed and valves rinsed in beaker filled with PBS.

3. Transferred each valve to 50 ml conical containing PBS. Washed by inversion and removed.

4. Repeated wash 3 times.

5. Dissected out the 3 cusps (valves).

6. Stored in PBS in 2 ml screw top Eppendorf Vials (Eppendorfs) and kept on ice.

Preparation of Stabilizers:

All stabilizers were prepared so that the final concentration of DMSO was 50%.

1 M Ascorbate in 50% DMSO:

Aldrich: cat#26,855-0, lot#10801HU 200 mg dissolved in 300 μl H₂O. Add 500 μl DMSO. The volume was adjusted to 1 ml with H₂O. Final pH was≈8.0.

1 M Coumaric Acid:

Sigma: cat#C-9008, lot#49H3600. MW 164.2

Dissolve 34.7 mg in 106 μl DMSO, pH≈3.0

138 μl H₂O was added. Sample precipitated out of solution.

Coumaric went back into solution once pH was adjusted to 7.5 with 1 N NaOH.

1 M n-Propyl Gallate:

Sigma: cat#P-3130, lot#117H0526. MW 212.2

Dissolve 58.2 mg in 138 μl DMSO.

Add 138 μl H₂O. Final pH is 6.5 or slightly lower.

Stabilizer Mixture (SM-a):

1.0 ml 500 mM Ascorbate

500 μl 1 M Coumaric Acid

300 μl 1 M n-propyl gallate

1.2 ml 50% DMSO

3.0 ml

Method:

1.6 ml of a solution (stabilizer mixture or PPG400) was added to each sample and then the sample was incubated at 4° C. for 2.5 days. Valves and 1 ml of the solution in which they were incubated were then transferred into 2 ml irradiation vials. Each sample was irradiated with gamma irradiation at a rate of 1.723 kGy/hr at 3.6° C. to a total dose of 25 kGy.

Hydrolysis of Tissue:

1. Washed each cusp 6 times with acetone in a 2 ml Eppendorf Vial.

2. After final acetone wash, dried sample in Savant (without heat) for approximately 10-15 minutes or until dry.

3. Weighed the samples, transferred them to hydrolysis vials and then added 6 N HCl at a volume of 20 mg tissue/ml HCl:

| Sample ID | Dry Weight (mg) | μl 6 N HCl |
|---|---|---|
| 1. PBS/0 kGy | 11.4 | 570 |
| 2. PBS/25 kGy | 6.0 | 300 |
| 3. DMSO/0 kGy | 6.42 | 321 |
| 4. DMSO/25 kGy | 8.14 | 407 |
| 5. DMSO/SM-a/0 kGy | 8.7 | 435 |
| 6. DMSO/SM-a/25 kGy | 8.15 | 408 |
| 7. PPG/0 kGy | 13.09 | 655 |
| 8. PPG/25 kGy | 10.88 | 544 |

SM=Stabilizer Mixture as defined above.

5. Samples were hydrolyzed at 110° C. for approximately 23 hours.

6. Hydrolysates were transferred into Eppendorf vials and centrifuged at 12,000 rpm for 5 min.

7. Supernatent was transferred into a clean Eppendorf vial.

8. 50 μl hydrolysate was diluted in 8 ml Nerl H₂O (diluting HCl to approximately 37 mM).

9. Spiked in 200 μl of 2× int-pyd. Mixed by inversion. (For 2000 μl 2× int-pyd: 200 μl 20×int-pyd+1.8 ml Nerl H₂O.)

10. Samples were loaded onto SP-Sephadex C25 column (approximately 1×1 cm packed bed volume) that had been equilibrated in water. (Column was pre-charged with NaCl)

11. Loaded flow through once again over column.

12. Washed with 20 ml 150 mM HCl.

13. Eluted crosslinks with 5 ml 2 N HCl into a low binding tube. 50 ml 2 N HCl:8.6 ml concentrated HCl adjusted to a volume of 50 ml with Nerl H₂O.

14. Dried entire sample in Savant.

Guanidine HCl Extraction and DEAE-Sepharose Purification of Proteoglycans:

4M Guianidine HCl Extraction:

1. Removed all three cusps from gamma irradiation vial and transferred to separate 50 ml conical tube.

2. Washed cusps five times with 50 ml dPBS (at 4° C. over approx. 5 hours) and determined wet weight of one cusp after drying on Kimwipe.

3. Transferred one cusp from each group to 1.5 ml microfuge tube and added appropriate volume of 4M guanidine HCl/150 mM sodium acetate buffer pH 5.8 with 2 μg/ml protease inhibitors (aprotinin, leupeptin, pepstatin A) to have volume to tissue ratio of 15 (see Methods in Enzymology Vol. 144 p.321—for optimal yield use ratio of 15 to 20).

4. Diced cusps into small pieces with scissors.

5. Nutated at 4° C. for ˜48 hours.

6. Centrifuged at 16,500 RPM on Hermle Z-252M, at 4° C. for 10 min.

7. Collected guanidine soluble fraction and dialyzed against PBS in 10K MWCO Slide-A-Lyzer overnight against 5 L PBS (3 slide-a-lyzers with one 5 L and 5 slide-a-lyzers in another 5 L) to remove guanidine.

8. Changed PBS and dialyzed for additional 9 hours at 4° C. with stirring.

9. Collected the dialysate and stored at 4° C.

10. Centrifuged at 16,500 RPM on Hermle Z-252M, at 4° C. for 5 min

11. Removed PBS soluble fraction for DEAE-Sepharose chromatography.

DEAE-Sepharose Chromatopography

1. Increased the NaCl concentration of 500 μl of PBS soluble guanidine extract to 300 mM NaCl (Assumed PBS soluble fractions were already at ˜150 mM NaCl, so added 15 μl 5M NaCl stock to each 500 μl sample).

2. Equilibrated ˜1 ml of packed DEAE-Sepharose (previously washed with 1M NaCl/PB pH 7.2) into 300 mM NaCl/PB pH 7.2 (Note: To make 300 mM NaCl/PB pH7.2- added 3 ml of 5M NaCl stock to 100 ml PBS).

3. Added 200 μl of 1:1 slurry of resin to 515 μL of GuHCl extracts (both at 300 mM NaCl).

4. Nutated at ambient temperature for ˜one hour.

5. Centrifuged gently to pellet resin.

6. Removed “unbound” sample and stored at ˜20° C.

7. Washed resin 5 times with ˜1.5 ml of 300 mM NaCl/PBS pH7.2.

8. After last wash, removed all extra buffer using a 100 μl Hamilton syringe.

9. Eluted at ambient temperature with three 100 μl volumes of 1M NaCl/PB pH7.2 and stored at −20° C.

SDS-PAGE:

5-20% gradient gels for analysis of PBS soluble Guanidine HCl extracts and DEAE-Sepharose chromatography.

1. Gel#1: GuHCl extracts/PBS soluble fractions—Toluidine blue and then Coomassie blue stained.

2. Gel#2: DEAE-Sepharose Eluant Fraction#1—Toluidine Blue stained then Coomassie Blue stained.

Quantification of Collagen Crosslinks by HPLC:

1. Prepared 100-200 μl 1× solution in 1% heptafluorobutyric acid (HFBA).

2. Injected 50 μl on C18 HPLC column equilibrated with mobile phase.

3. Spectrofluorometer was set for excitation at 295 nm and emission at 395 nm.

4. Calculated the integrated fluorescence of Internal-Pyridinoline (Int-Pyd) per 1 μl of 1× solution of Int-Pyd.

Results:

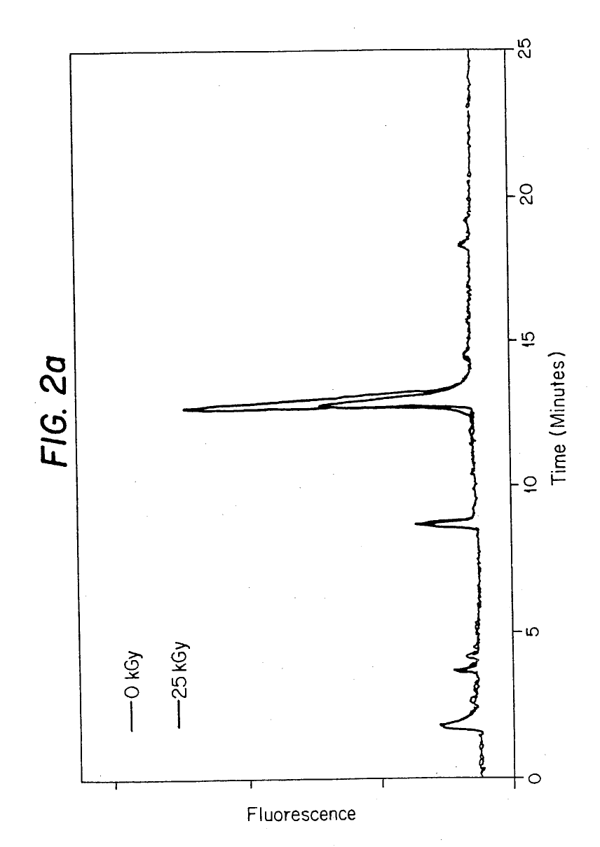

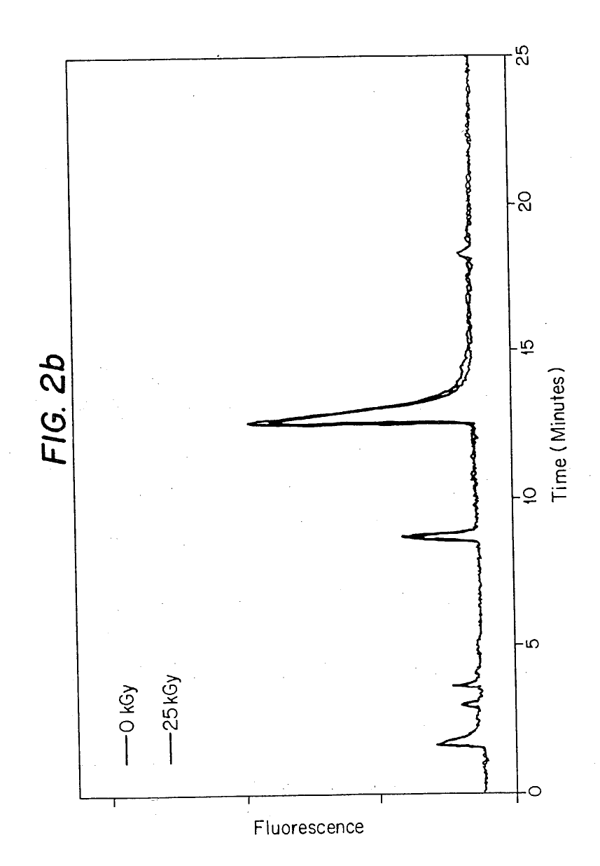

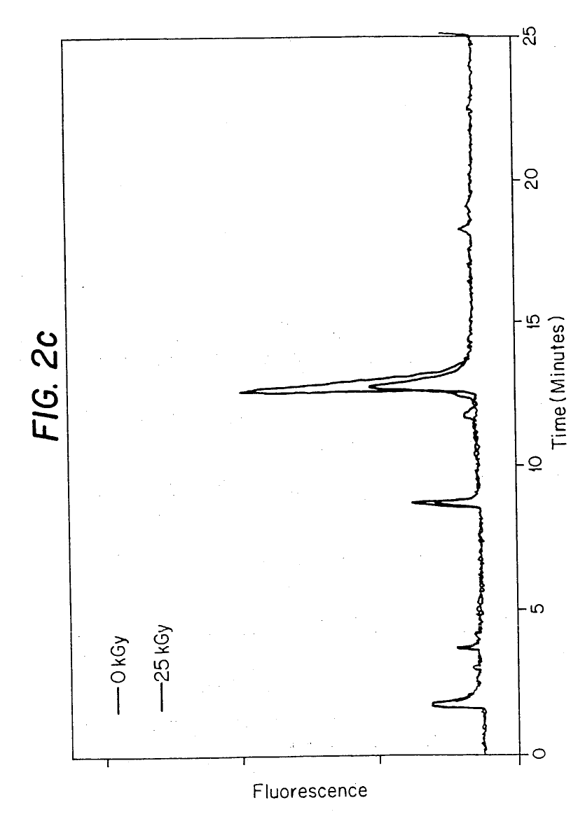

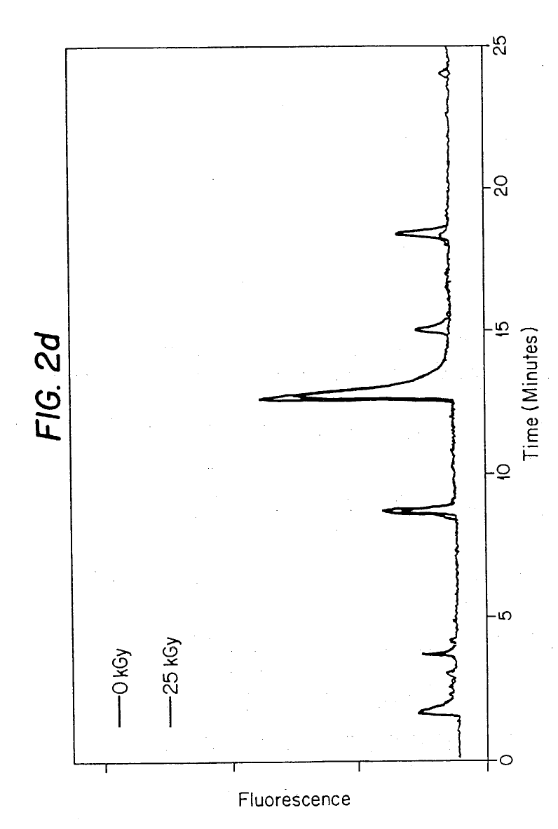

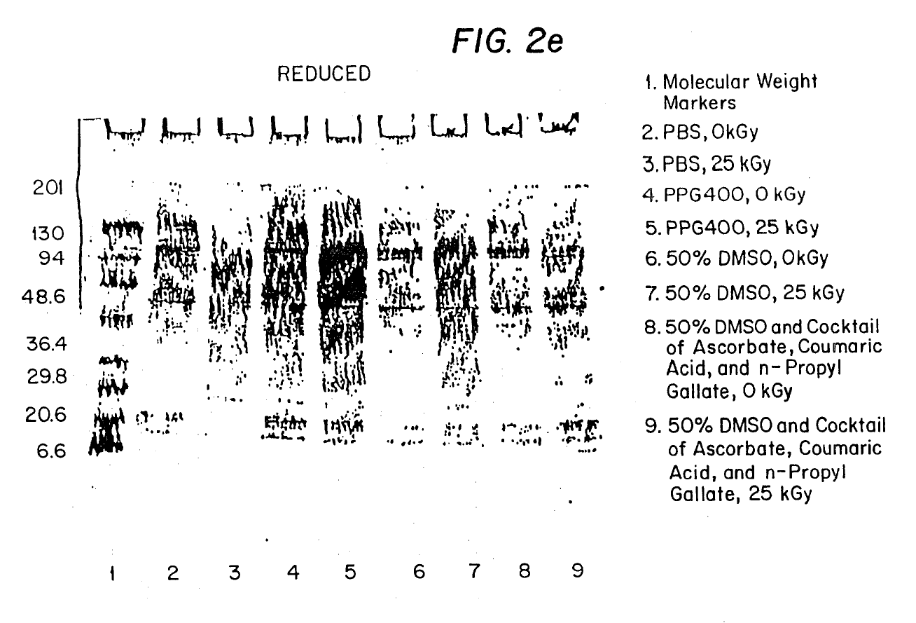

The HPLC results are shown in FIGS. 2A-D. The major peak represents the Internal-Pyridinoline (int-Pyd) peak. Irradiation in an aqueous environment (PBS) produced pronounced decreases in the smaller peaks (FIG. 2A). Reduction of the water content by the addition of a non-aqueous solvent (PPG 400) produced a nearly superimposable curve (FIG. 2B). DMSO was less effective (FIG. 2C), while DMSO plus a mixture of stabilizers (FIG. 2D) was more effective at preserving the major peak although some minor peaks increased somewhat. The area under the pyd peak for each sample was calculated as shown in the table below. These results confirm the above conclusions and show that the amino acid crosslinks (pyd) found in mature collagen are effectively conserved in the samples containing PPG and DMSO with a scavenger mixture. Gel analysis is shown in FIG. 2E and reflects the major conclusions from the HPLC analysis, with significant loss of bands seen in PBS and retention of the major bands in the presence of non-aqueous solvents.

| Sample | Area of Pyd Peak |
|---|---|
| PBS/0 kGy | 94346 |
| PBS/25 kGy | 60324 |
| DMSO/0 kGy | 87880 |
| DMSO/25 kGy | 49030 |
| DMSO/SM/0 kGy | 75515 |
| DMSO/SM/25 kGy | 88714 |
| PPG/0 kGy | 99002 |
| PPG/25 kGy | 110182 |

### Example 3 [071001alm071gamma]

In this experiment, frozen porcine AV heart valves soaked in various solvents were gamma irradiated to a total dose of 30 kGy at 1.584 kGy/hr at −20° C.

Materials:

1. Porcine heart valve cusps were obtained and stored at −80° C. in a cryopreservative solution (Containing Fetal calf serum, Penicillin-Streptomycin. M199 media, and approximately 20% DMSO).

2. Dulbecco's Phosphate Buffered Saline. Gibco BRL: cat#14190-144. lot#1095027

3. 2 ml screw cap vials. VWR: cat#20170-221, lot#0359

4. 2 ml glass vials. Wheaton: cat#223583, lot#370000-01

5. 13 mm stoppers. Stelmi: 6720GC, lot#G006/5511

6. DMSO. JT Baker: cat#9224-01, lot#H40630

7. Sodium ascorbate. Aldrich: cat#26,855-0, lot 10801HU; prepared as a 2M stock in Nerl water.

8. Fetal calf serum

9. Penicillin-Streptomycin

10. M199 media

11. DMSO

Methods:

Cryopreservative Procedure:

Preparation of Solutions

Freeze Medium:

Fetal calf serum (FCS) (10%)=50 ml

Penicillin-Streptomycin=2.5 ml

M199=QS 500 ml

2DMSO

DMSO=15.62 g

Freeze Medium =QS 100 ml

3M DMSO

DMSO=23.44 g

Freeze Medium=QS 100 ml

Preparation of Tissue

1. Placed dissected heart valves (with a small amount of conduit/muscle attached) into glass freezing tubes (label with pencil).

2. Added 2 ml of freeze medium.

3. At 21° C., added 1 ml 2M DMSO solution.

4. At 5 minutes, added 1 ml 2M DMSO solution.

5. At 30 minutes, added 4 ml 3M DMSO solution.

6. At 45 minutes and 4° C., placed freezing tubes on ice.

7. At 50 minutes and −7.2° C., seeded bath, which is an alcohol filled tank inside the cryopreservation machine and is used to lower the temperature quickly.

8. At 55 minutes and −7.2° C., nucleated. Nucleation is a processing step that allows the tissue to freeze evenly and quickly without much ice formation. This is done by placing a steel probe in a liquid nitrogen canister, touching the probe to the outside of the freezing tube at the surface of the solution, waiting for ice formation, shaking the tube and placing the tube in the bath.

9. At 70 minutes, cooled to −40° C. at 1° C./minute. Removed from bath and placed in canister of liquid N₂, and stored in cryogenic storage vessel.

Procedure for Irradiation of Heart Valves:

1. Thawed AV heart valve cusps on wet ice.

2. Pooled cusps into 50 ml tubes.

3. Washed cusps with ˜50 ml dPBS at 4° C. while nutating. Changed PBS 5 times over the course of 5 hrs.

4. Transferred cusps into 2 ml screw cap tubes (2 cusps/tube).

5. Added 1.0 ml of the following to two of each of two tubes: dPBS, 50% DMSO and 50% DMSO with 200 mM sodium ascorbate (2M sodium ascorbate stock was diluted as follows: 400 μl (2M)+1.6 ml water+2 ml 100% DMSO).

6. Incubated tubes at 4° C. with nutating for ˜46 hours.

7. Transferred solutions and cusps to glass 2 ml vials, stoppered and capped.

8. All vials were frozen on dry ice.

9. Frozen samples were then irradiated at −20° C. at a rate of 1.584 kGy/hr to a total dose of 30 kGy.

Results:

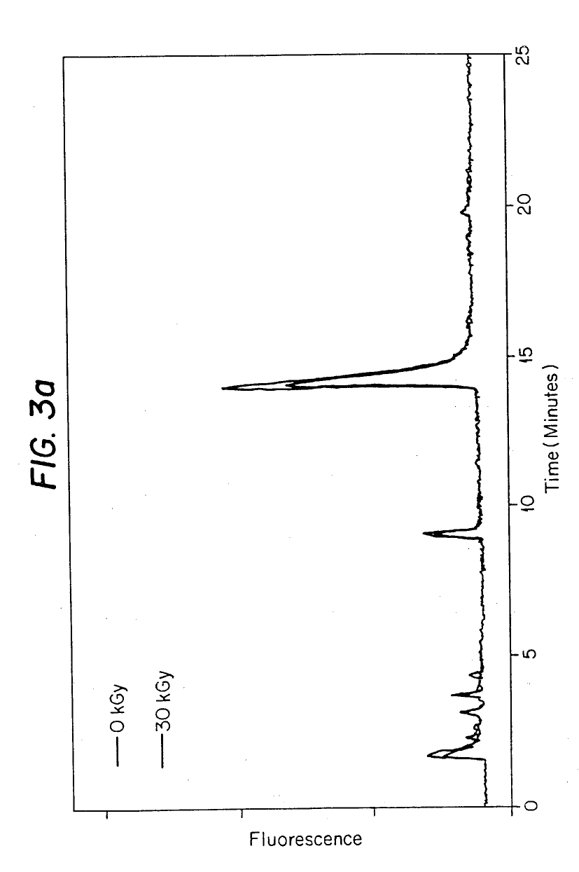

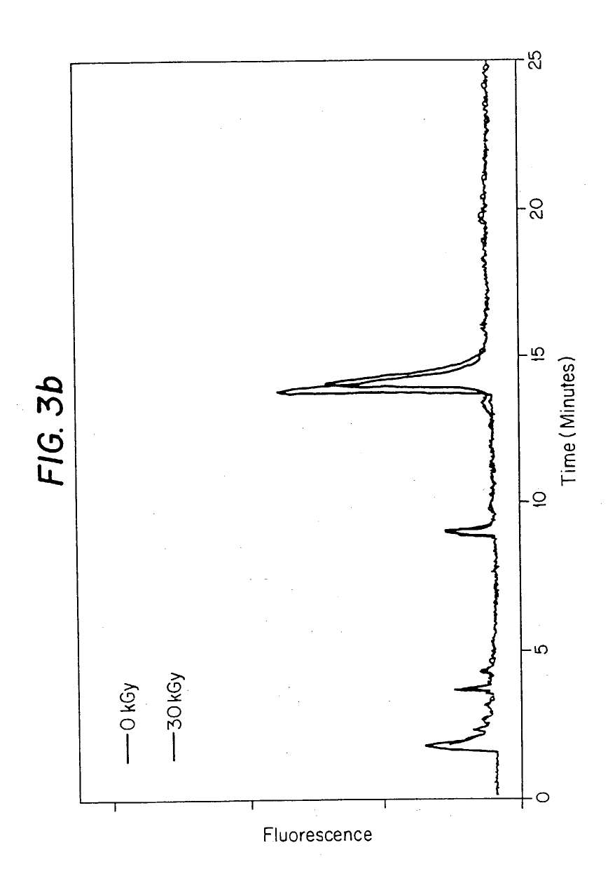

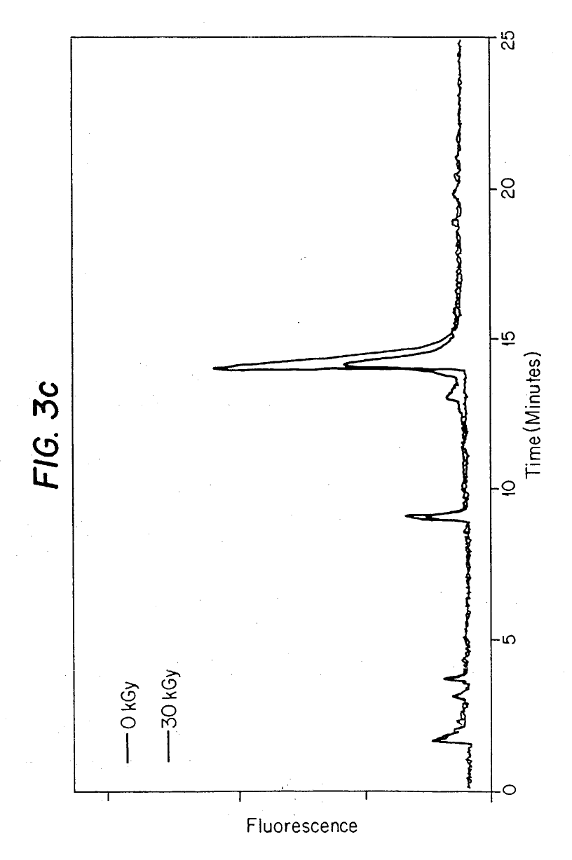

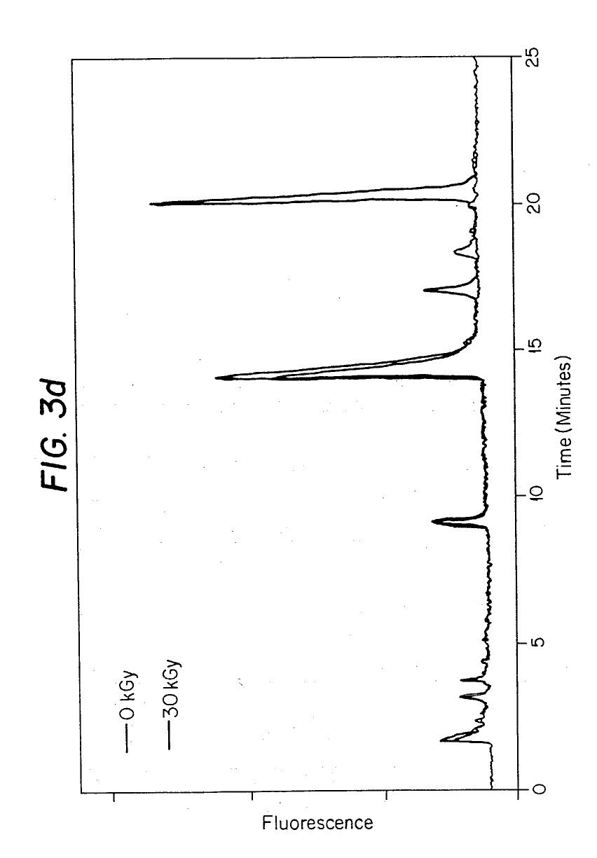

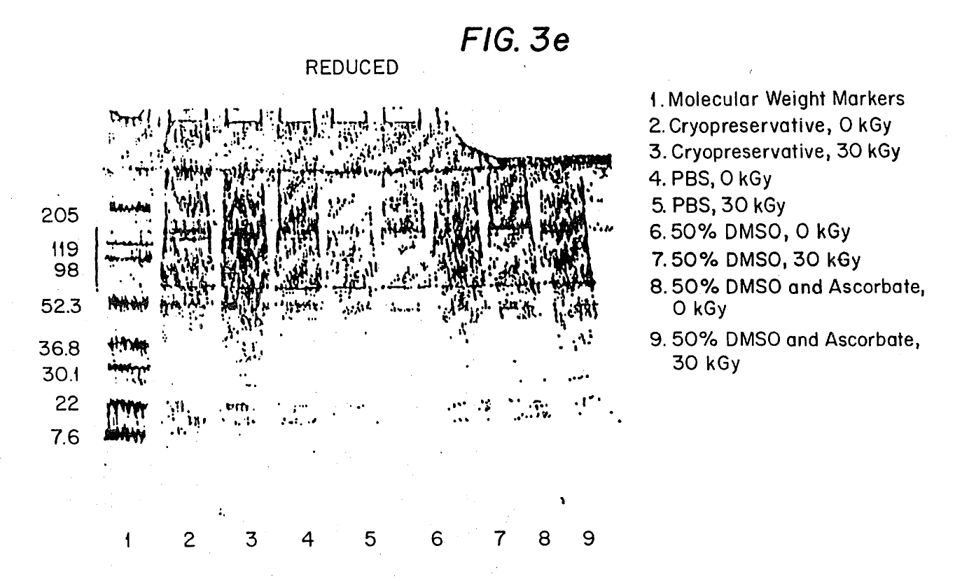

The results of the HPLC analysis are shown in FIGS. 3A-3D. Irradiation in an aqueous environment (PBS) produced decreases in the smaller peaks (FIG. 3A). Reduction of the water content by the addition of a non-aqueous solvent (20% DMSO) reproduced these peaks more faithfully (FIG. 3B). Increasing the DMSO concentration to 50% was slightly more effective (FIG. 3C), while DMSO plus a mixture of stabilizers (FIG. 3D) was very effective at preserving both the major and minor peaks (the additional new peaks are due to the stabilizers themselves). Gel analysis is shown in FIG. 3E and reflects the major conclusions from the HPLC analysis, with significant loss of bands seen in PBS and retention of the major bands in the presence of non-aqueous solvents with and without stabilizers.

### Example 4 [072001alm073gamma]

In this experiment, frozen porcine AV heart valves soaked in various solvents were gamma irradiated to a total dose of 45 kGy at approximately 6 kGy/hr at −70° C.

Materials:

1. Porcine heart valve cusps were obtained and stored at −80° C. in a cryopreservative solution (Same solution as that in Example 3).

2. Dulbecco's Phosphate Buffered Saline (dPBS). Gibco BRL: cat#14190-144, lot 1095027

3. 2 ml screw cap vials. VWR: cat#20170-221, lot#0359

4. 2 ml glass vials. Wheaton: cat#223583, lot#370000-01

5. 13 mm stoppers. Stelmi: 6720GC, lot#G006/5511

6. DMSO. JT Baker: cat#9224-01, lot#H40630

7. Sodium ascorbate. Aldrich: cat#26,855-0, lot 10801HU; prepared as a 2M stock in Nerl water.

8. Polypropylene glycol 400 (PPG400). Fluka: cat#81350, lot#386716/1

Methods:

Cryopreservative Procedure is the Same as that Shown in Example 3.

1. Thawed AV heart valve cusps on wet ice. Dissected out cusps and washed the pooled cusps 6 times with cold PBS.

2. Dried each cusp and transferred cusps into 2 ml screw cap tubes (2 cusps/tube).

3. Added 1.2 ml of the following to two of each of two tubes: dPBS, dPBS with 200 mM sodium ascorbate, PPG400, PPG400 for rehydration, 50% DMSO and 50% DMSO with 200 mM sodium ascorbate (2M sodium ascorbate stock was diluted as follows: 400 μl (2M)+1.6 ml water+2 ml 100% DMSO).

4. Incubated tubes at 4° C. with nutating for ˜46 hours.

5. Replaced all solutions with fresh solutions (with the following exception: for one PPG400 set, PPG400 was removed, the cusp washed with PBS+200 mM ascorbate, which was then removed and replaced with fresh PBS+200 mM ascorbate).

6. Incubated tubes at 4° C. with nutating for ˜46 hours.

7. Changed the solution on the PPG400 dehyd./PBS+ascorbate rehydration cusps prepared in step 5.

8. Incubated tubes at 4° C. with nutating for 6 hours.

9. Transferred solutions and cusps to glass 2 ml vials, stoppered and capped.

10. All vials were frozen on dry ice.

11. Frozen samples were then irradiated at −70° C. at a rate of 6 kGy/hr to a total dose of 45 kGy.

Results:

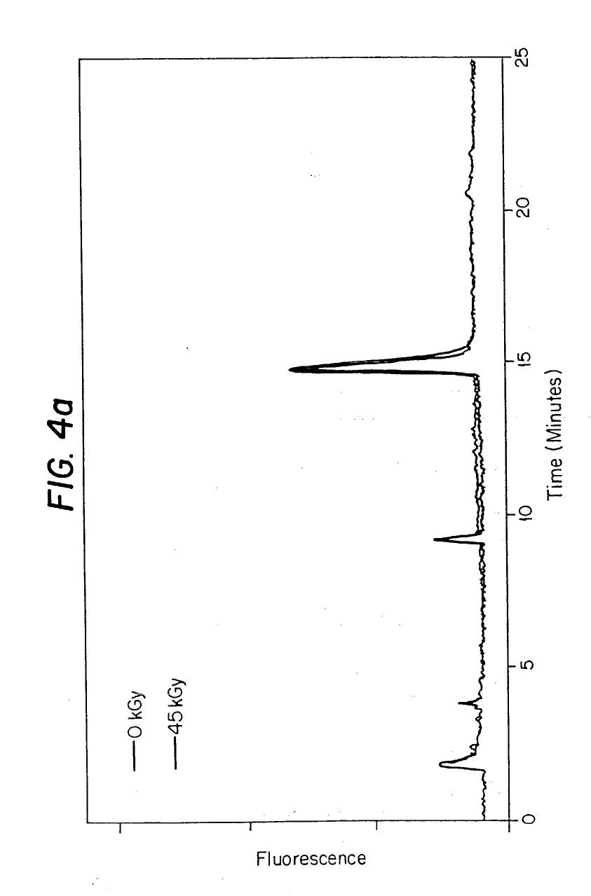

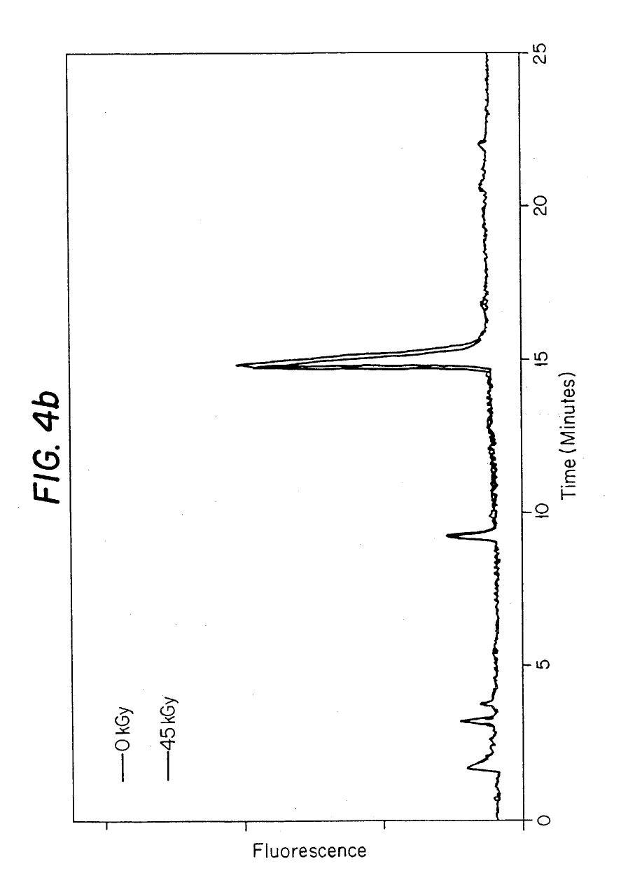

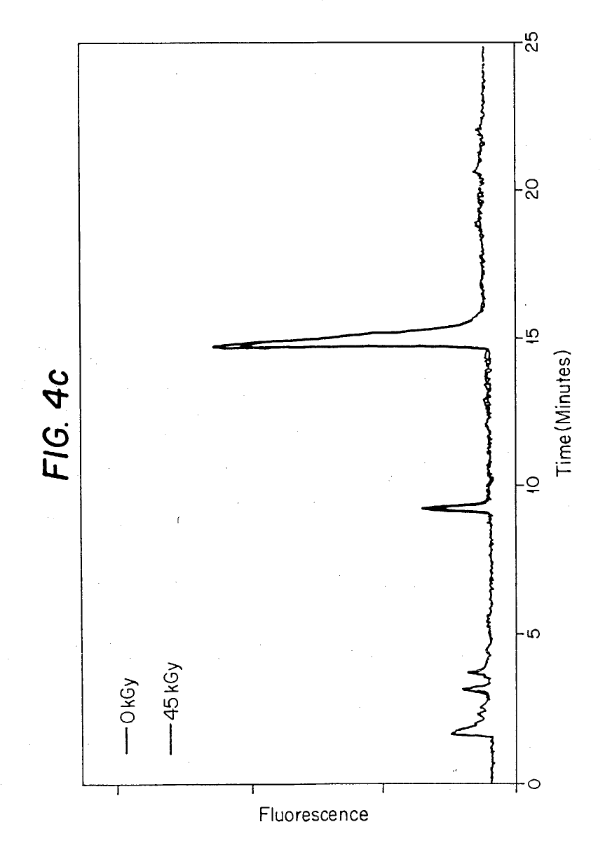

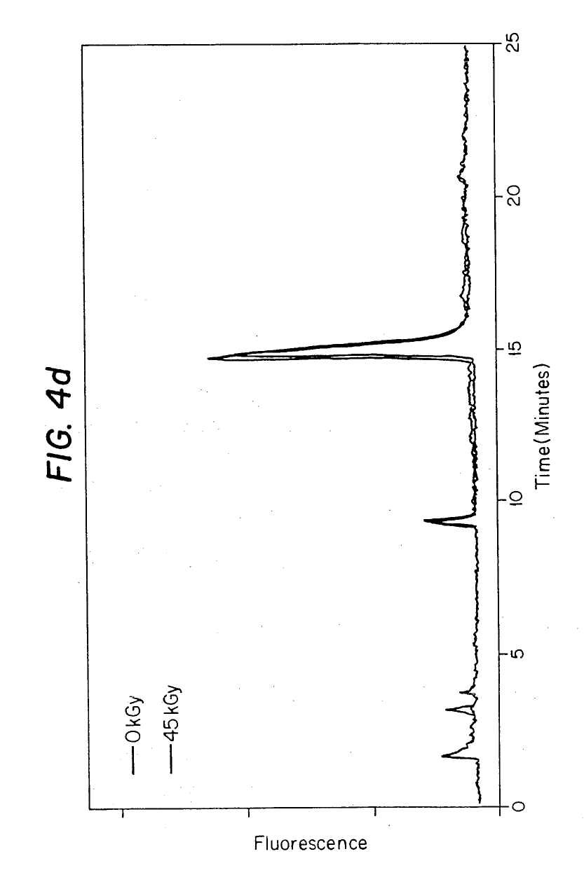

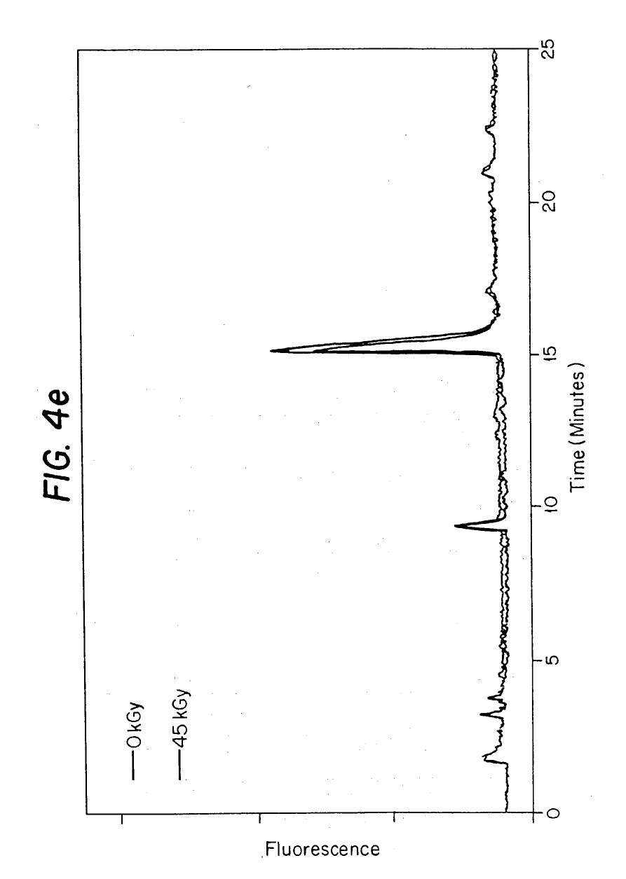

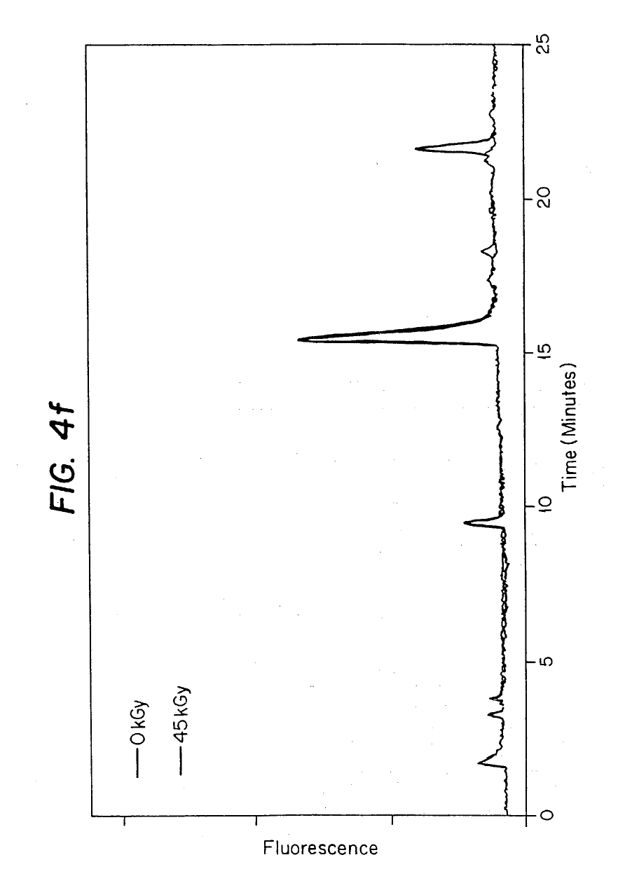

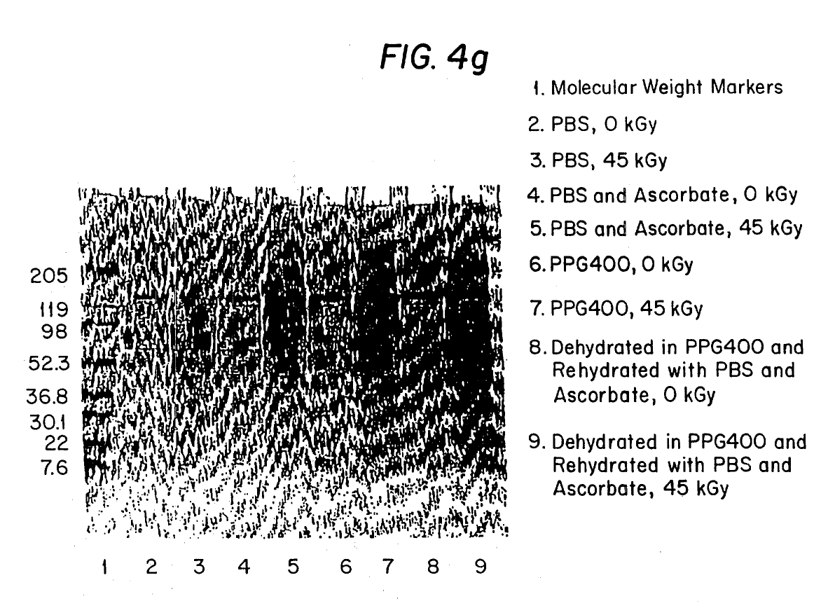

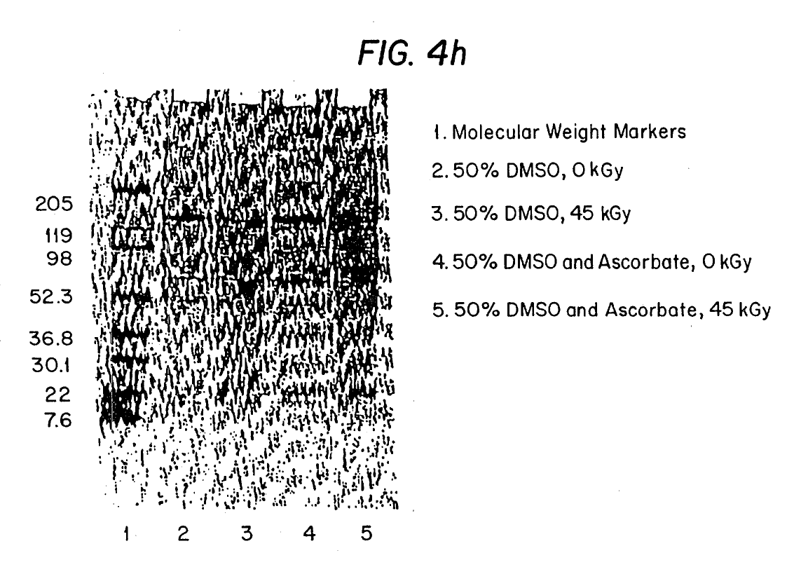

The results of the HPLC analysis are shown in FIGS. 4A-4F. Irradiation in an aqueous environment (PBS) resulted in changes in the minor peaks and a right shift in the major peak. The inclusion of various non-aqueous solvents, reduction in residual water, and the addition of stabilizers produced profiles that more closely matched those of the corresponding controls. The gel analysis is shown in FIGS. 4G-4H and shows a significant loss of bands in PBS, while the other groups demonstrated a significant retention of these lost bands.

When comparing the results from Example 4 to the results from Examples 1, 2, and 3, it becomes apparent that lowering the temperature for the gamma irradiation usually results in a decrease in the amount of modification or damage to the collagen crosslinks. One illustration of this temperature dependence is the sample containing 50% DMSO and ascorbate, in which the additional peaks are markedly decreased as the temperature is lowered from −20° C. to −80° C. It is also clear that reducing residual water content by replacing it with a non-aqueous solvent results in less damage or modification, as does adding the stabilizers shown.

Having now fully described this invention, it will be understood to those of ordinary skill in the art that the methods of the present invention can be carried out with a wide and equivalent range of conditions, formulations and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

What is claimed is:

1. A method for sterilizing one or more heart valves that are sensitive to radiation, said method comprising irradiating said one or more heart valves with radiation for a time effective to sterilize said one or more heart valves at a rate effective to sterilize said one or more heart valves and to protect said one or more heart valves from said radiation.

2. A method for sterilizing one or more heart valves that are sensitive to radiation, said method comprising:
(i) applying to said one or more heart valves at least one stabilizing process selected from the group consisting of:
(a) adding to said one or more heart valves at least one stabilizer in an amount effective to protect said one or more heart valves from said radiation;
(b) reducing the residual solvent content of said one or more heart valves to a level effective to protect said one or more heart valves from said radiation;
(c) reducing the temperature of said one or more heart valves to a level effective to protect said one or more heart valves from said radiation;
(d) reducing the oxygen content of said one or more heart valves to a level effective to protect said one or more heart valves from said radiation;
(e) adjusting the pH of said one or more heart valves to a level effective to protect said one or more heart valves from said radiation; and
(f) adding to said one or more heart valves at least one non-aqueous solvent in an amount effective to protect said one or more heart valves from said radiation; and
(ii) irradiating said one or more heart valves with a suitable radiation at an effective rate for a time effective to sterilize said one or more heart valves.

3. A method for sterilizing one or more heart valves that are sensitive to radiation, said method comprising:
(i) applying to said one or more heart valves at least one stabilizing process selected from the group consisting of:
(a) adding to said one or more heart valves at least one stabilizer;
(b) reducing the residual solvent content of said one or more heart valves;
(c) reducing the temperature of said one or more heart valves;
(d) reducing the oxygen content of said one or more heart valves;
(e) adjusting the pH of said one or more heart valves; and
(f) adding to said one or more heart valves at least one non-aqueous solvent; and
(ii) irradiating said one or more heart valves with a suitable radiation at an effective rate for a time effective to sterilize said one or more heart valves, wherein said at least one stabilizing process and the rate of irradiation are together effective to protect said one or more heart valves from said radiation.

4. A method for sterilizing one or more heart valves that are sensitive to radiation, said method comprising:
(i) applying to said one or more heart valves at least two stabilizing processes selected from the group consisting of:
(a) adding to said one or more heart valves at least one stabilizer;
(b) reducing the residual solvent content of said one or more heart valves;
(c) reducing the temperature of said one or more heart valves;
(d) reducing the oxygen content of said one or more heart valves;
(e) adjusting the pH of said one or more heart valves; and
(f) adding to said one or more heart valves at least one non-aqueous solvent; and
(ii) irradiating said one or more heart valves with a suitable radiation at an effective rate for a time effective to sterilize said one or more heart valves, wherein said at least two stabilizing processes are together effective to protect said one or more heart valves from said radiation and further wherein said at least two stabilizing processes may be performed in any order.

5. The method according to claim 2, 3 or 4, wherein said residual solvent is an organic solvent.

6. The method according to claim 1, 2, 3 or 4, wherein said effective rate is not more than about 3.0 kGy/hour.

7. The method according to claim 1, 2, 3 or 4, wherein said effective rate is not more than about 2.0 kGy/hr.

8. The method according to claim 1, 2, 3 or 4, wherein said effective rate is not more than about 1.0 kGy/hr.

9. The method according to claim 1, 2, 3 or 4, wherein said effective rate is not more than about 0.3 kGy/hr.

10. The method according to claim 1, 2, 3 or 4, wherein said effective rate is more than about 3.0 kGy/hour.

11. The method according to claim 1, 2, 3 or 4, wherein said effective rate is at least about 6.0 kGy/hour.

12. The method according to claim 1, 2, 3 or 4, wherein said effective rate is at least about 18.0 kGy/hour.

13. The method according to claim 1, 2, 3 or 4, wherein said effective rate is at least about 30.0 kGy/hour.

14. The method according to claim 1, 2, 3 or 4, wherein said effective rate is at least about 45 kGy/hour.

15. The method according to claim 1, 2, 3 or 4, wherein said one or more heart valves is maintained in a low oxygen atmosphere.

16. The method according to claim 1, 2, 3 or 4, wherein said one or more heart valves is maintained in an atmosphere comprising at least one noble gas or nitrogen.

17. The method according to claim 16, wherein said noble gas is argon.

18. The method according to claim 1, 2, 3 or 4, wherein said one or more heart valves is maintained in a vacuum.

19. The method according to claim 2, 3 or 4, wherein said residual solvent content is reduced by a method selected from the group consisting of lyophilization, drying, concentration, addition of a second solvent, evaporation, chemical extraction, spray-drying and vitrification.

20. The method according to claim 2, 3 or 4, wherein said residual solvent content is less than about 15%.

21. The method according to claim 2, 3 or 4, wherein said residual solvent content is less than about 10%.

22. The method according to claim 2, 3 or 4, wherein said residual solvent content is less than about 3%.

23. The method according to claim 2, 3 or 4, wherein said residual solvent content is less than about 2%.

24. The method according to claim 2, 3 or 4, wherein said residual solvent content is less than about 1%.

25. The method according to claim 2, 3 or 4, wherein said residual solvent content is less than about 0.5%.

26. The method according to claim 2, 3 or 4, wherein said residual solvent content is less than about 0.08%.

27. The method according to claim 1, 2, 3 or 4, wherein at least one sensitizer is added to said one or more heart valves prior to said step of irradiating said one or more heart valves.

28. The method according to claim 1, 2, 3, or 4, wherein said one or more heart valves contains at least one biological contaminant or pathogen selected from the group consisting of viruses, bacteria, yeasts, molds, fungi, parasites and prions or similar agents responsible, alone or in combination, for TSEs.

29. The method according to claim 2, 3 or 4, wherein said at least one stabilizer is an antioxidant.

30. The method according to claim 2, 3 or 4, wherein said at least one stabilizer is a free radical scavenger or spin trap.

31. The method according to claim 2, 3 or 4, wherein said at least one stabilizer is a combination stabilizer.

32. The method according to claim 2, 3 or 4, wherein said at least one stabilizer is a ligand.

33. The method according to claim 32, wherein said ligand is heparin.

34. The method according to claim 2, 3 or 4, wherein said at least one stabilizer reduces damage due to reactive oxygen species.

35. The method according to claim 2, 3 or 4, wherein said at least one stabilizer is selected from the group consisting of: ascorbic acid or a salt or ester thereof; glutathione; vitamin E or a derivative thereof, including Trolox; albumin; sucrose; glycylglycine; L-carnosine; cysteine; silymarin; diosmin; hydroquinonesulfonic acid; 6-hydroxy-2,5,7,8-tetramethylchroman-2-carboxylic acid; uric acid or a salt or ester thereof; methionine; histidine; N-acetyl cysteine; lipoic acid; sodium formaldehyde sulfoxylate; gallic acid or a derivative thereof; propyl gallate; ethanol; acetone; rutin; epicatechin; biacalein; purpurogallin; coumaric acid; and mixtures of two or more thereof.

36. The method according to claim 35, wherein said mixtures of two or more stabilizers are selected from the group consisting of: mixtures of ethanol and acetone; mixtures of ascorbic acid, or a salt or ester thereof, and uric acid, or a salt or ester thereof; mixtures of ascorbic acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, 6-hydroxy-2,5,7,8-tetramethylchroman-2-carboxylic acid, and albumin; mixtures of ascorbic acid, or a salt or ester thereof, uric acid, or a salt or ester thereof, 6-hydroxy-2,5,7,8-tetramethylchroman-2-carboxylic acid, albumin and sucrose; mixtures of ascorbic acid, or a salt or ester thereof, and glycylglycine; mixtures of ascorbic acid, or a salt or ester thereof, glycylglycine and albumin; mixtures of ascorbic acid, or a salt or ester thereof, and L-carnosine; mixtures of ascorbic acid, or a salt or ester thereof, and cysteine; mixtures of ascorbic acid, or a salt or ester thereof, and N-acetyl cysteine; mixtures of ascorbic acid, or a salt or ester thereof, uric acid, or a salt or ester thereof, 6-hydroxy-2,5,7,8-tetramethylchroman-2-carboxylic acid, and silymarin; mixtures of ascorbic acid, or a salt or ester thereof, uric acid, or a salt or ester thereof, 6-hydroxy-2,5,7,8-tetramethylchroman-2-carboxylic acid, and diosmin; mixtures of ascorbic acid, or a salt or ester thereof, uric acid, or a salt or ester thereof, and lipoic acid; mixtures of ascorbic acid, or a salt or ester thereof, uric acid, or a salt or ester thereof, and hydroquinonesulfonic acid; mixtures of Trolox, α-lipoic acid, coumaric acid and n-propyl gallate; and mixtures of uric acid, or a salt or ester thereof, lipoic acid, sodium formaldehyde sulfoxylate, gallic acid, or a derivative thereof, propyl gallate; and 6-hydroxy-2,5,7,8-tetramethylchroman-2-carboxylic acid.

37. The method according to claim 2, 3 or 4, wherein said at least one stabilizer is a dipeptide stabilizer.

38. The method according to claim 37, wherein said dipeptide stabilizer is selected from the group consisting of glycyl-glycine (Gly-Gly), carnosine and anserine.

39. The method according to claim 1, 2, 3 or 4, wherein said radiation is corpuscular radiation, electromagnetic radiation, or a mixture thereof.

40. The method according to claim 39, wherein said electromagnetic radiation is selected from the group consisting of radio waves, microwaves, visible and invisible light, ultraviolet light, x-ray radiation, gamma radiation and combinations thereof.

41. The method according to claim 1, 2, 3 or 4, wherein said radiation is gamma radiation.

42. The method according to claim 1, 2, 3 or 4, wherein said radiation is E-beam radiation.

43. The method according to claim 1, 2, 3 or 4, wherein said radiation is visible light.

44. The method according to claim 1, 2, 3 or 4, wherein said radiation is ultraviolet light.

45. The method according to claim 1, 2, 3 or 4, wherein said radiation is x-ray radiation.

46. The method according to claim 1, 2, 3 or 4, wherein said radiation is polychromatic visible light.

47. The method according to claim 1, 2, 3 or 4, wherein said radiation is infrared.

48. The method according to claim 1, 2, 3 or 4, wherein said radiation is a combination of one or more wavelengths of visible and ultraviolet light.

49. The method according to claim 1, 2, 3 or 4, wherein said irradiation is conducted at ambient temperature.

50. The method according to claim 1, 2, 3 or 4, wherein said irradiation is conducted at a temperature below ambient temperature.

51. The method according to claim 1, 2, 3 or 4, wherein said irradiation is conducted below the freezing point of at least one or more solvents within or surrounding said one or more heart valves.

52. The method according to claim 1, 2, 3 or 4, wherein said irradiation is conducted below the eutectic point of at least one or more solvents within or surrounding said one or more heart valves.

53. The method according to claim 1, 2, 3 or 4, wherein said irradiation is conducted at a temperature above ambient temperature.

54. A composition comprising one or more heart valves and at least one stabilizer in an amount effective to preserve said one or more heart valves for their intended use following sterilization with radiation.

55. A composition comprising one or more heart valves, wherein the residual solvent content of said one or more heart valves is at a level effective to preserve said one or more heart valves for their intended use following sterilization with radiation.

56. The composition of claim 55, wherein said residual solvent content is less than about 15%.

57. The composition of claim 55, wherein said residual solvent content is less than about 10%.

58. The composition of claim 55, wherein said residual solvent content is less than about 5%.

59. The composition of claim 55, wherein said residual solvent content is less than about 2%.

60. The composition of claim 55, wherein said residual solvent content is less than about 1%.

61. The composition of claim 55, wherein said residual solvent content is less than about 0.5%.

62. The composition of claim 55, wherein said residual solvent content is less than about 0.08%.

63. The composition of claim 54 or 55, wherein said one or more heart valves is glassy or vitrified.

64. The method according to claim 2, 3 or 4, wherein said non-aqueous solvent is selected from the group consisting of glycerol, DMSO, ethanol, acetone, PPG, and mixtures thereof.

65. The method according to claim 64, wherein said PPG is PPG 400, PPG 1200 or PPG 2000.

66. The method according to claim 2, 3 or 4, wherein said residual solvent content is about 0%.

67. The method according to claim 2, 3 or 4, wherein said residual solvent content is about 1%.

68. The method according to claim 2, 3 or 4, wherein said residual solvent content is about 2.4%.

69. The method according to claim 2, 3 or 4, wherein said residual solvent content is about 4.8%.

70. The method according to claim 2, 3 or 4, wherein said residual solvent content is about 7%.

71. The method according to claim 2, 3 or 4, wherein said residual solvent content is about 9%.

72. The method according to claim 2, 3 or 4, wherein said residual solvent content is about 10%.

73. The method according to claim 2, 3 or 4, wherein said residual solvent content is about 20%.

74. The method according to claim 2, 3 or 4, wherein said residual solvent content is about 33%.

75. The method according to claim 2, 3 or 4, wherein said residual solvent content is less than about 33%.

76. The composition of claim 54, wherein said at least one stabilizer is selected from the group consisting of: ascorbic acid or a salt or ester thereof; glutathione; vitamin E or a derivative thereof; albumin; Trolox; coumaric acid; sucrose; glycylglycine; L-carnosine; cysteine; silymarin; diosmin; hydroquinonesulfonic acid; 6-hydroxy-2,5,7,8-tetramethylchroman-2-carboxylic acid; uric acid or a salt or ester thereof; methionine; histidine; N-acetyl cysteine; lipoic acid; sodium formaldehyde sulfoxylate; gallic acid or a derivative thereof; propyl gallate; ethanol; acetone; rutin; epicatechin; biacalein; purpurogallin; and mixtures of two or more thereof.

77. The composition according to claim 76, wherein said mixtures of two or more stabilizers are selected from the group consisting of: mixtures of ethanol and acetone; mixtures of ascorbic acid, or a salt or ester thereof, and uric acid, or a salt or ester thereof; mixtures of ascorbic acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, and 6-hydroxy-2,5,7,8-tetramethylchroman-2-carboxylic acid; mixtures of ascorbic acid, or a salt or ester thereof, uric acid, or a salt or ester thereof, 6-hydroxy-2,5,7,8-tetramethylchroman-2-carboxylic acid, and albumin; mixtures of ascorbic acid, or a salt or ester thereof, uric acid, or a salt or ester thereof, 6-hydroxy-2,5,7,8-tetramethylchroman-2-carboxylic acid, albumin and sucrose; mixtures of ascorbic acid, or a salt or ester thereof and glycylglycine; mixtures of ascorbic acid, or a salt or ester thereof, glycylglycine and albumin; mixtures of ascorbic acid, or a salt or ester thereof, and L-carnosine; mixtures of ascorbic acid, or a salt or ester thereof, and cysteine; mixtures of ascorbic acid, or a salt or ester thereof, and N-acetyl cysteine; mixtures of ascorbic acid, or a salt or ester thereof, uric acid, or a salt or ester thereof, 6-hydroxy-2,5,7,8-tetramethylchroman-2-carboxylic acid, and silymarin; mixtures of ascorbic acid, or a salt or ester thereof, uric acid, or a salt or ester thereof, 6-hydroxy-2,5,7,8-tetramethylchroman-2-carboxylic acid, and diosmin; mixtures of ascorbic acid, or a salt or ester thereof, uric acid, or a salt or ester thereof, and lipoic acid; mixtures of ascorbic acid, or a salt or ester thereof, uric acid, or a salt or ester thereof, and hydroquinonesulfonic acid; mixtures of Trolox, α-lipoic acid, and coumaric acid; mixtures of Trolox, α-lipoic acid, coumaric acid and n-propyl gallate; and mixtures of uric acid, or a salt or ester thereof, lipoic acid, sodium formaldehyde sulfoxylate, gallic acid, or a derivative thereof, propyl gallate, and 6-hydroxy-2,5,7,8-tetramethylchroman-2-carboxylic acid.

78. A method for prophylaxis or treatment of a condition or disease in a mammal comprising introducing into a mammal in need thereof one or more heart valves stabilized according to a method of one of claims 1, 2, 3 or 4.

79. The method according to claim 2, 3 or 4, wherein said residual solvent is an aqueous solvent.

80. The method according to claim 2, 3 or 4, wherein said one or more heart valves is suspended in said solvent.

81. The method according to claim 1, 2, 3 or 4, wherein said irradiation is conducted below the glass transition point of at least one or more solvents within or surrounding said one or more heart valves.

82. The method according to claim 1, 2, 3 or 4, wherein the recovery of the desired characteristic(s) or composition of the one or more heart valves after sterilization by irradiation is greater than 100% of the pre-irradiation value.

83. The method according to claim 1, 2, 3 or 4, wherein the recovery of the desired characteristic(s) or composition of the one or more heart valves after sterilization by irradiation is at least about 100% of the pre-irradiation value.

84. The method according to claim 1, 2, 3, 4 or 11, wherein the recovery of the desired activity of the one or more heart valves after sterilization by irradiation is at least about 90% of the pre-irradiation value.

85. The method according to claim 1, 2, 3, 4 or 11, wherein the recovery of the desired activity of the one or more heart valves after sterilization by irradiation is at least about 80% of the pre-irradiation value.

86. The method according to claim 1, 2, 3, 4 or 11, wherein the recovery of the desired activity of the one or more heart valves after sterilization by irradiation is at least about 70% of the pre-irradiation value.

87. The method according to claim 1, 2, 3, 4 or 11, wherein the recovery of the desired activity of the one or more heart valves after sterilization by irradiation is at least about 60% of the pre-irradiation value.

88. The method according to claim 1, 2, 3, 4 or 11, wherein the recovery of the desired activity of the one or more heart valves after sterilization by irradiation is at least about 50% of the pre-irradiation value.

89. One or more heart valves prepared according to a method of one of claims 1, 2, 3 or 4.

90. The method according to claim 2, 3 or 4, wherein said residual solvent content is less than about 80%.

91. The method according to claim 2, 3 or 4, wherein said residual solvent content is less than about 50%.

92. The composition of claim 55, wherein said residual solvent content is less than about 80%.

93. The composition of claim 55, wherein said residual solvent content is less than about 50%.

94. A composition comprising one or more heart valves, at least one non-aqueous solvent and at least one stabilizer in an amount effective to preserve said one or more heart valves for their intended use following sterilization with radiation.

95. The composition of claim 94, wherein said at least one non-aqueous solvent comprises DMSO and said at least one stabilizer comprises ascorbate.

96. The composition of claim 94, wherein said at least one non-aqueous solvent comprises DMSO and said at least one stabilizer comprises a mixture of ascorbate, coumaric acid and n-propyl gallate.

97. The composition of claim 94, wherein said at least one non-aqueous solvent comprises PPG and said at least one stabilizer comprises ascorbate.

98. The method according to claim 4, wherein, said at least two stabilizing processes comprise:
a. adding to said one or more heart valves at least one stabilizer; and
b. adding to said one or more heart valves at least one non-aqueous solvent.

99. The method according to claim 98, wherein said at least one non-aqueous solvent comprises DMSO and said at least one stabilizer comprises ascorbate.

100. The method according to claim 98, wherein said at least one non-aqueous solvent comprises DMSO and said at least one stabilizer comprises a mixture of ascorbate, Coumaric acid and n-propyl gallate.

101. The method according to claim 98, wherein said at least one non-aqueous solvent comprises PPG and said at least one stabilizer comprises ascorbate.

102. The method according to claim 2, 3 or 4, wherein the residual solvent is a mixture of an organic solvent and an aqueous solvent.

103. A composition comprising one or more heart valves and at least one stabilizer, wherein the residual solvent content of said one or more heart valves is at a level that together with said at least one stabilizer is effective to preserve said one or more heart valves for their intended use following sterilization with radiation.

104. The composition according to claim 54, 55 or 103, wherein the oxygen content of said one or more heart valves is reduced to a level that together with said at least one stabilizer and/or said residual solvent content is effective to protect said one or more heart valves from with radiation.
